(19) United States
(12) Patent Application Publication
(10) Pub. No.: US 2025/0291247 A1
(43) Pub. Date: Sep. 18, 2025

(54) AMINE COMPOUND, RESIST COMPOSITION INCLUDING THE SAME, AND METHOD OF FORMING PATTERN BY USING THE RESIST COMPOSITION

(71) Applicant: Samsung Electronics Co., Ltd., Suwon-si (KR)

(72) Inventors: Changki KIM, Suwon-si (KR); Cheol KANG, Suwon-si (KR); Chanjae AHN, Suwon-si (KR); Hyeran KIM, Suwon-si (KR); Jeongho MUN, Suwon-si (KR); Honggu IM, Hwaseong-si (KR); Sungwon CHOI, Suwon-si (KR)

(73) Assignee: Samsung Electronics Co., Ltd., Suwon-si (KR)

(21) Appl. No.: 18/828,296

(22) Filed: Sep. 9, 2024

(30) Foreign Application Priority Data
Mar. 13, 2024 (KR) 10-2024-0035436

(51) Int. Cl. G03F 7/004; C07D 285/14; C07D 293/10; C07D 333/36; C07D 345/00; C07D 495/04; C07D 495/14; C07D 517/04; C09D 7/63; C09D 133/10; G03F 7/039; H01L 21/027

(52) U.S. Cl. CPC G03F 7/0045; C07D 285/14; C07D 293/10; C07D 333/36; C07D 345/00; C07D 495/04; C07D 495/14; C07D 517/04; C09D 7/63; C09D 133/10; G03F 7/0043; G03F 7/0392; G03F 7/0397; H01L 21/0274

(57) ABSTRACT

Provided are an amine compound represented by Formula 1, a resist composition including the same, and a method of forming a pattern by using the same:

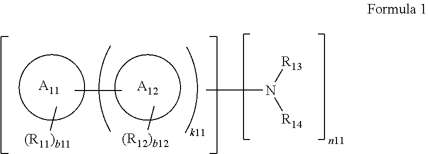

wherein detailed descriptions of A₁₁, A₁₂, k₁₁, n₁₁, R₁₁ to R₁₄, b11 and b12 in Formula 1 are provided in the present specification.

### CROSS-REFERENCE TO RELATED APPLICATION

This application is based on and claims priority under 35 U.S.C. § 119 to Korean Patent Application No. 10-2024-0035436, filed on Mar. 13, 2024, in the Korean Intellectual Property Office, the disclosure of which is incorporated by reference herein in its entirety.

### BACKGROUND

### 1. Field

The disclosure relates to an amine compound, a resist composition including the same, and a method of forming a pattern by using the resist composition.

### 2. Description of the Related Art

In semiconductor manufacturing, resists have physical properties that change in response to light, and are used to form fine patterns. From among these resists, chemically amplified resists have been widely used. A chemically amplified resist enables patterning by changing the solubility of a base resin in a developer by reaction of an acid, which is formed by a reaction between light and a photoacid generator, with the base resin again.

In particular, when high-energy rays with relatively very high energy such as extreme ultraviolet (EUV) are used, there is a problem in that the number of photons may be significantly small even when irradiating light of the same energy. Accordingly, there may be a demand for a resist composition that can act effectively even when a small amount is used and that can provide improved sensitivity, improved resolution, and/or reduced defects.

### SUMMARY

Provided are an amine compound capable of providing improved sensitivity, improved resolution, and/or reduced defects, a resist composition including the amine compound, and a method of forming a pattern by using the resist composition.

Additional aspects will be set forth in part in the description which follows and, in part, will be apparent from the description, or may be learned by practice of the presented embodiments of the disclosure.

According to an aspect of the disclosure, provided is an amine compound represented by Formula 1:

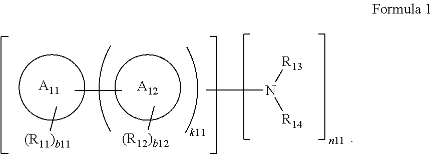

In Formula 1, N is nitrogen,
A₁₁ may be one of i) a first ring, ii) a condensed ring in which at least two first rings are condensed with each other, or iii) a condensed ring in which at least one first ring and at least one second ring are condensed with each other,
A₁₂ may be one of i) a single bond, ii) a first ring, iii) a second ring, iv) a condensed ring in which at least two first rings are condensed with each other, v) a condensed ring in which at least two second rings are condensed with each other, or vi) a condensed ring in which at least one first ring and at least one second ring are condensed each other,
the first ring may be a 5-membered ring including at least one of S, Se, and Te, and the second ring may be a 4- to 10-membered ring optionally including a heteroatom,
k11 may be an integer from 0 to 3,
n11 may be an integer from 1 to 4,
R₁₁ and R₁₂ may each independently be at least one of hydrogen, deuterium, a halogen, a cyano group, a nitro group, a hydroxyl group, a carboxylate group, an ester moiety, a substituted or unsubstituted C₁-C₃₀ alkyl group, a substituted or unsubstituted C₃-C₃₀ cycloalkyl group, a substituted or unsubstituted C₁-C₃₀ alkoxy group, or a substituted or unsubstituted C₃-C₃₀ cycloalkoxy group,
R₁₃ and R₁₄ may each independently be a substituted or unsubstituted C₆-C₃₀ aryl group or a substituted or unsubstituted C₁-C₃₀ heteroaryl group, and
b11 and b12 may each independently be an integer from 1 to 8.

According to another aspect of the disclosure, a resist composition includes the amine compound, a polymer, a photoacid generator, and an organic solvent.

According to another aspect of the disclosure, a method of forming a pattern includes forming a resist film by applying the resist composition, exposing at least a portion of the resist film to high energy rays to provide an exposed resist film, and developing the exposed resist film by using a developer.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a pattern formation method according to at least one embodiment;

FIGS. 2A to 2C are each a side cross-sectional view illustrating a pattern formation method according to at least one embodiment;

FIGS. 3A to 3E are each a cross-sectional view illustrating a method of forming a patterned structure, according to at least one embodiment; and

FIGS. 4A to 4E are each a cross-sectional view illustrating a method of manufacturing a semiconductor device, according to at least one embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figure, to explain aspects. As used herein, the term “and/or” includes any and all combinations of one or more of the associated listed items. Expressions such as “at least one of,” when preceding a list of elements, modify the entire list of elements and do not modify the individual elements of the list.

As embodiments allow for various changes and numerous embodiments, example embodiments will be illustrated in the drawings and described in detail in the written description. However, this is not intended to limit embodiments to particular modes of practice, and it is to be appreciated that all changes, equivalents, and substitutes that do not depart from the spirit and technical scope of the disclosure are encompassed in embodiments. In the description of embodiments certain detailed explanations of the related art are omitted when it is deemed that they may unnecessarily obscure the essence of the disclosure.

Terms such as “first”, “second”, “third”, and the like may be used to describe various components, but are used only for the purpose of distinguishing one component from other components, and the order, type, and the like of the components are not limited.

It will be understood that when a component, such as a layer, a film, a region, or a plate, is referred to as being “on” or “above” another component in the specification, the component can directly contact to be above, below, right, or left of the another component as well as being above, below, left, or right of the another component in a non-contact manner.

An expression used in the singular encompasses the expression of the plural, unless it has a clearly different meaning in the context. It is to be understood that the terms such as “including,” “having,” and “comprising” are intended to indicate the existence of the features, numbers, steps, actions, components, parts, ingredients, materials, or combinations thereof disclosed in the specification, and are not intended to preclude the possibility that one or more other features, numbers, steps, actions, components, parts, ingredients, materials, or combinations thereof may exist or may be added.

Whenever a range of values is enumerated, the range includes all values within the range as if recorded explicitly clearly, and may further include the boundaries of the range. Accordingly, the range of “X” to “Y” includes all values between X and Y, including X and Y. Additionally, when the terms “about” or “substantially” are used in this specification in connection with a numerical value and/or geometric terms, it is intended that the associated numerical value includes a manufacturing tolerance (e.g., ±10%) around the stated numerical value. Further, regardless of whether numerical values and/or geometric terms are modified as “about” or “substantially,” it will be understood that these values should be construed as including a manufacturing or operational tolerance (e.g., ±10%) around the stated numerical values and/or geometry.

The term “Cₓ-C_{y}” as used herein refers to a case where the number of carbons constituting the substituent is x to y. For example, the term “C₁-C₆” refers to a case where the number of carbons constituting the substituent is 1 to 6, and the term “C₆-C₂₀” refers to a case where the number of carbons constituting the substituent is 6 to 20.

The term “monovalent hydrocarbon group” as used herein refers to a monovalent residue derived from an organic compound including carbon and hydrogen or a derivative of the organic compound, and examples thereof may include: a linear or branched alkyl group (e.g., a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cyclic aliphatic hydrocarbon group e.g., a cycloalkyl group) (e.g., a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, a norbornyl group, a norbornylmethyl group, a tricyclotricyclodecanyl group, a tetracyclododecanyl group, a tetracyclododecanylmethyl group, and a dicyclohexylmethyl group); a monovalent unsaturated aliphatic hydrocarbon group (e.g., an alkenyl group, an alkynyl group, and an allyl group); a monovalent unsaturated cyclic aliphatic hydrocarbon group (e.g., a cycloalkenyl group and a 3-cyclohexenyl group); an aryl group (e.g., a phenyl group, a 1-napthyl group, and a 2-napthyl group); an arylalkyl group (e.g., a benzyl group and a diphenylmethyl group); a heteroatom-containing monovalent hydrocarbon group (e.g., a tetrahydrofuranyl group, a methoxymethyl group, an ethoxy methyl group, a methylthiomethyl group, an acetamidemethyl group, a trifluoroethyl group, a (2-methoxyethoxy)methyl group, an acetoxymethyl group, a 2-carboxyl-1-cyclohexyl group, a 2-oxopropyl group, a 4-oxo-1-adamantyl group, and a 3-oxocyclohexyl group); or any combination thereof. In addition, among these groups, some hydrogen atoms may be substituted with a moiety including a heteroatom, such as oxygen, sulfur, nitrogen, or a halogen atom, or some carbon atoms may be replaced by a moiety including a heteroatom, such as oxygen, sulfur, or nitrogen. Accordingly, these groups may each include a hydroxyl group, a cyano group, a carbonyl group, a carboxyl group, an ether linkage, an ester linkage, a sulfonate ester linkage, a carbonate, a lactone ring, a sultone ring, a carboxylic anhydride moiety, or a haloalkyl moiety.

The term “divalent hydrocarbon group” as used herein refers to a divalent residue in which one hydrogen of the monovalent hydrocarbon group is replaced by a binding site to a neighboring atom. Examples of the divalent hydrocarbon group may include a linear or branched alkylene group, a cycloalkylene group, an alkenylene group, an alkynylene group, a cycloalkylene group, an arylene group, or a group in which some carbon atoms are replaced by a heteroatom.

The term “alkyl group” as used herein refers to a linear or branched monovalent saturated aliphatic hydrocarbon group, and examples thereof may include a methyl group, an ethyl group, a propyl group, an isobutyl group, a sec-butyl group, a ter-butyl group, a pentyl group, an iso-amyl group, a hexyl group, and the like. The term “alkylene group” as used herein refers to a linear or branched divalent saturated aliphatic hydrocarbon group, and examples thereof may include a methylene group, an ethylene group, a propylene group, a butylene group, an isobutyl group, and the like.

The term “halogenated alkyl group” as used herein refers to a group in which one or more hydrogen atoms of an alkyl group are substituted with halogen, and examples include may include CF₃ and the like.

The term “alkoxy group” as used herein refers to a monovalent group represented by-OA₁₀₁, wherein A₁₀₁ is an alkyl group. Examples thereof may include a methoxy group, an ethoxy group, an isopropyloxy group, and the like.

The term “alkylthio group” as used herein refers to a monovalent group represented by -SA₁₀₁, wherein A₁₀₁ is an alkyl group.

The term “halogenated alkyl group” as used herein refers to a group in which one or more hydrogen atoms of an alkoxy group are substituted with halogen, and examples thereof may include —OCF₃ and the like.

The term “halogenated alkylthio group” as used herein refers to a group in which one or more hydrogen atoms of an alkylthio group are substituted with halogen, and examples thereof may include —SCF₃ and the like.

The term “cycloalkyl group” as used herein refers to a monovalent saturated hydrocarbon cyclic group, and examples thereof may include a monocyclic group, such as a cyclopropyl group, a cyclobutyl group, a cyclopentyl group, a cyclohexyl group, a cycloheptyl group, and the like, and a polycyclic condensed group, such as a norbornyl group and an adamantyl group. The term “cycloalkylene group” used herein refers to a divalent saturated hydrocarbon cyclic group, and examples thereof include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, a dicyclohexylmethylene group, and the like.

The term “cycloalkoxy group” as used herein refers to a monovalent group represented by —OA₁₀₂, wherein A₁₀₂ is a cycloalkyl group. Examples thereof may include a cyclopropoxy group, a cyclobutoxy group, and the like.

The term “cycloalkylthio group” as used herein refers to a monovalent group represented by -SA₁₀₂, wherein A₁₀₂ is a cycloalkyl group.

The term “heterocycloalkyl group” as used herein refers to a group in which some carbon atoms of the cycloalkyl group are replaced by a moiety including a heteroatom, such as oxygen, sulfur, or nitrogen, and examples of the heterocycloalkyl group include an ether linkage, an ester linkage, a sulfonic ester linkage, a carbonate, a lactone ring, a sultone ring, or a carboxylic anhydride moiety. The term “heterocycloalkylene group” as used herein refers to a group in which some carbon atoms of the cycloalkylene group are replaced by a moiety including a heteroatom, such as oxygen, sulfur, or nitrogen.

The term “heterocycloalkoxy group” as used herein refers to a monovalent group represented by-OA₁₀₃, wherein A₁₀₃ is a heterocycloalkyl group.

The term “alkenyl group” as used herein refers to a linear or branched monovalent unsaturated aliphatic hydrocarbon including one or more carbon-carbon double bond. The term “alkenylene group” as used herein refers to a linear or branched divalent unsaturated aliphatic hydrocarbon including one or more carbon-carbon double bonds.

The term “alkenyloxy group” as used herein refers to a monovalent group represented by —OA₁₀₄, wherein A₁₀₄ is an alkenyl group.

The term “cycloalkenyl group” as used herein refers to a monovalent unsaturated cyclic hydrocarbon group including one or more carbon-carbon double bonds. The term “cycloalkenylene group” as used herein refers to a divalent unsaturated cyclic hydrocarbon group including one or more carbon-carbon double bonds.

The term “cycloalkenyloxy group” as used herein refers to a monovalent group represented by-OA₁₀₅, wherein A₁₀₅ is an alkenyl group.

The term “heterocycloalkenyl group” as used herein refers to a group in which some carbon atoms of the cycloalkenylene group are replaced by a moiety including a heteroatom, such as oxygen, sulfur, or nitrogen. The term “heterocycloalkenylene group” as used herein refers to a group in which some carbon atoms of the cycloalkenylene group are replaced by a moiety including a heteroatom, such as oxygen, sulfur, or nitrogen.

The term “heterocycloalkenyloxy group” as used herein refers to a monovalent group represented by-OA₁₀₆, wherein A₁₀₆ is a heterocycloalkenyl group.

The term “alkynyl group” as used herein refers to a linear or branched unsaturated aliphatic monovalent hydrocarbon including one or more carbon-carbon triple bonds.

The term “alkynyloxy group” as used herein refers to a monovalent group represented by —OA₁₀₇, wherein A₁₀₇ is an alkenyl group.

The term “aryl group” as used herein refers to a monovalent group having a carbocyclic aromatic system, and examples thereof may include a phenyl group, a naphthyl group, an anthracenyl group, a phenanthrenyl group, a pyrenyl group, a chrysenyl group, and the like.

The term “aryloxy group” as used herein refers to a monovalent group represented by-OA₁₀₈, wherein A₁₀₈ is an alkyl group.

The term “heteroaryl group” as used herein refers to a monovalent group having a heterocyclic aromatic system, and examples thereof may include a pyridinyl group, a pyrimidinyl group, a pyrazinyl group, and the like. The term “heteroarylene group” as used herein refers to a divalent group having a heterocyclic aromatic system.

The term “heteroaryloxy group” as used herein refers to a monovalent group represented by —OA₁₀₉, wherein A₁₀₉ is a heteroaryl group.

The term “substituent” as used herein substitution of, e.g., a hydrogen, and may include: deuterium, halogen, a hydroxyl group, a cyano group, a nitro group, a carbonyl group, a carboxylate group, an amino group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, or a C₁-C₂₀ heteroarylthio group, each substituted with deuterium, halogen, a hydroxyl group, a cyano group, a nitro group, a carbonyl group, a carboxylate group, an amino group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, and/or any combination thereof.

Hereinafter, the disclosure will be described in detail by explaining embodiments with reference to the accompanying drawings, and substantially identical or corresponding components are given the same reference numerals in the drawings, and thus a description thereof will be omitted. In the drawings, thicknesses are magnified to clearly illustrate various layers and regions. Also in the diagrams, thicknesses of some layers and regions are exaggerated for convenience of explanation. Meanwhile, embodiments described below are illustrative examples of embodiments, and various changes in forms and details may be made.

### [Amine Compound]

An aspect of the disclosure provides an amine compound represented by Formula 1:

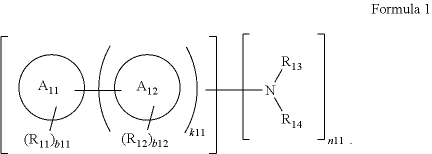

In Formula 1,
A₁₁ may be i) a first ring, ii) a condensed ring in which at least two first rings are condensed with each other, or iii) a condensed ring in which at least one first ring and at least one second ring are condensed with each other,
A₁₂ may be i) a first ring, ii) a second ring, iii) a condensed ring in which at least two first rings are condensed with each other, iv) a condensed ring in which at least two second rings are condensed with each other, or v) a condensed ring in which at least one first ring and at least one second ring are condensed each other,
the first ring may be a 5-membered ring including at least one of S, Se, and Te, and the second ring is a 4- to 10-membered ring optionally including a heteroatom,
k11 may be an integer from 0 to 3,
n11 may be an integer from 1 to 4,
R₁₁ and R₁₂ may each independently be hydrogen, deuterium, a halogen, a cyano group, a nitro group, a hydroxyl group, a carboxylate group, an ester moiety, a substituted or unsubstituted C₁-C₃₀ alkyl group, a substituted or unsubstituted C₃-C₃₀ cycloalkyl group, a substituted or unsubstituted C₁-C₃₀ alkoxy group, and/or a substituted or unsubstituted C₃-C₃₀ cycloalkoxy group,
R₁₃ and R₁₄ may each independently be a substituted or unsubstituted C₆-C₃₀ aryl group and/or a substituted or unsubstituted C₁-C₃₀ heteroaryl group, and
b11 and b12 may each independently be an integer from 1 to 8. Additionally, in the case wherein K11 is 0, A₁₂ may be a single bond between the nitrogen (N) and A₁₁.

In at least one example, the first ring may be represented by Formula 4:

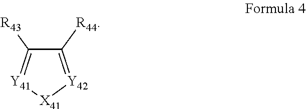

In Formula 4,
X₄₁ may be S, Se, or Te,
Y₄₁ may be N or CR₄₁,
Y₄₂ may be N or CR₄₂,
R₄₁ to R₄₄ may each independently be *—N(R₁₃)(R₁₄), hydrogen, deuterium, a halogen, a cyano group, a nitro group, a hydroxyl group, a carboxylate group, an ester moiety, a substituted or unsubstituted C₃-C₃₀ alkyl group, a substituted or unsubstituted C₃-C₃₀ cycloalkyl group, a substituted or unsubstituted C₃-C₃₀ alkoxy group, or a substituted or unsubstituted C₃-C₃₀ cycloalkoxy group, and

R₁₃ and R₁₄ may each independently be a substituted or unsubstituted C₆-C₃₀ aryl group or a substituted or unsubstituted C₁-C₃₀ heteroaryl group.

For example, the first ring may be represented by one of Formulae 4-1 to 4-6:

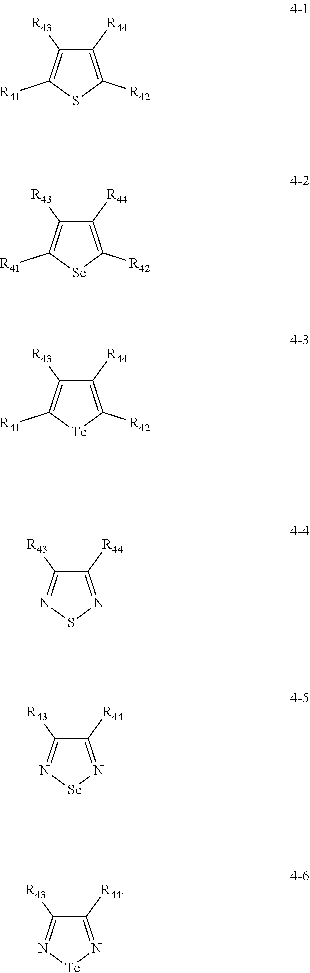

In Formulae 4-1 to 4-6, descriptions of R₄₁ to R₄₄ are referred to the present specification.

In at least one example, the second ring may be tetrahydropyrane, dihydropyrane, pyrane, tetrahydrothiopyrane, dihydrothiopyrane, thiopyrane, tetrahydrofurane, dihydrofurane, piperidine, tetrahydropyridine, dihydropyridine, pyrrolidine, dihydropyrrole, pyrrole, imidazole, pyrazole, furan, oxazole, pyridine, pyrazine, pyridazine, pyrimidine, triazine, cyclopentane, cyclopentadiene, cyclohexane, cyclohexene, cyclohexadiene, benzene, naphthalene, and/or the like.

For example, the second ring may be at least one of pyrrole, furan, pyridine, pyrazine, pyridazine, pyrimidine, triazine, benzene, or naphthalene.

For example, in Formula 1, A₁₁ and A₁₂ may each independently be i) the first ring, ii) a condensed ring in which at least two of the first rings are condensed with each other, or iii) a condensed ring in which at least one of the first ring and at least one second ring are condensed with each other. A₁₁ and A₁₂ may be the same and/or different from each other.

In detail, in Formula 1, A₁₁ and A₁₂ may each independently be represented by one of Formulae 4-11 to 4-26:

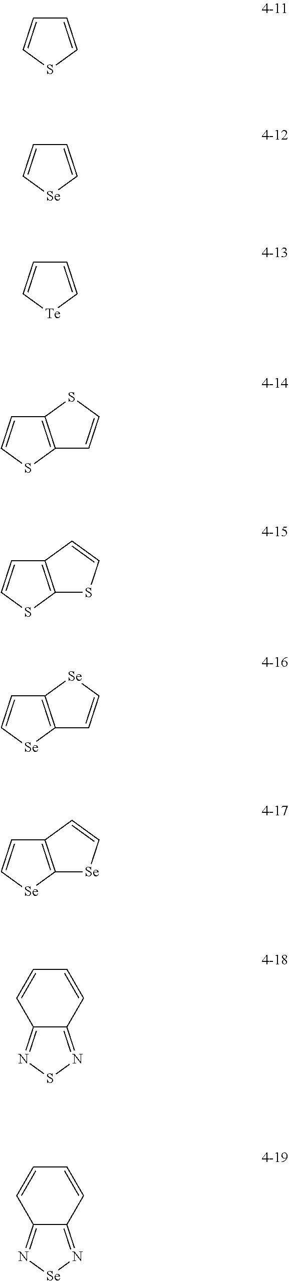

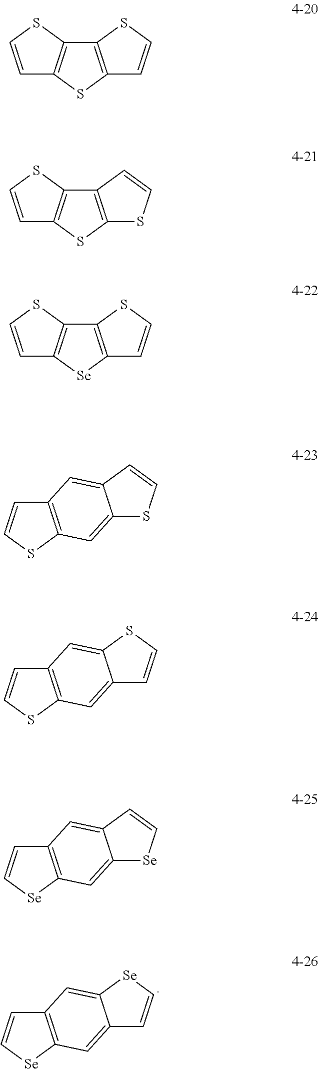

In Formula 1, k11 indicates the number of A₁₂(s), and for example, k11 in Formula 1 may be 0 or 1.

In Formula 1, n11 indicates the number of —N(R₁₃)(R₁₄)(s), and for example, n11 in Formula 1 may be 1 or 2.

For example, in Formula 1, R₁₁ and R₁₂ may each independently be selected from: hydrogen; deuterium; a halogen; a hydroxyl group; a carboxylate group; an ester moiety; and a C₁-C₃₀ alkyl group, a C₃-C₃₀ cycloalkyl group, a C₁-C₃₀ alkoxy group, and a C₃-C₃₀ cycloalkoxy group, each unsubstituted or substituted with deuterium, a halogen, a hydroxyl group, a carboxylate group, an ester moiety, and/or a combination thereof.

For example, in Formula 1, R₁₃ and R₁₄ may each independently be a monovalent group based on a substituted or unsubstituted first ring, a monovalent group based on a substituted or unsubstituted second ring, a monovalent group based on a substituted or unsubstituted condensed ring in which at least two of the first ring are condensed with each other, a monovalent group based on a substituted or unsubstituted condensed ring in which at least of the two second ring are condensed with each other, and/or a monovalent group based on a substituted or unsubstituted condensed ring in which at least one of the first ring and at least one of the second ring are condensed with each other.

In at least one embodiment, in Formula 1, i) R₁₃ and R₁₄ may each independently be a monovalent group based on a substituted or unsubstituted second ring, and/or a monovalent group based on a substituted or unsubstituted condensed ring in which at least two second rings are condensed with each other; ii) R₁₃ may be a monovalent group based on a substituted or unsubstituted first ring, a monovalent group based on a substituted or unsubstituted condensed ring in which at least two first rings are condensed with each other, and/or a monovalent group based on a substituted or unsubstituted condensed ring in which at least one first ring and at least one second ring are condensed with each other, and R₁₄ may be a monovalent group based on a substituted or unsubstituted second ring, and/or a monovalent group based on a substituted or unsubstituted condensed ring in which at least two second rings are condensed with each other; or iii) R₁₃ and R₁₄ may each independently be a monovalent group based on a substituted or unsubstituted first ring, a monovalent group based on a substituted or unsubstituted condensed ring in which at least two first rings are condensed with each other, and/or a monovalent group based on a substituted or unsubstituted condensed ring in which at least one first ring and at least one second ring are condensed with each other.

In at least one embodiment, in Formula 1, R₁₃ and R₁₄ may each independently be a monovalent group based on a substituted or unsubstituted second ring, or a monovalent group based on a substituted or unsubstituted condensed ring in which at least two second rings are condensed with each other.

For example, in Formula 1, R₁₃ and R₁₄ may each independently be a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, benzofuranyl group, benzothiophenyl group, isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, a halogen, a hydroxyl group, a carboxylate group, an ester moiety, a C₁-C₂₀ alkyl group, a C₁-C₂₀ alkoxy group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, and/or a combination thereof.

In at least one embodiment, in Formula 1, R₁₃ and R₁₄ may each independently be a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a cinnolinyl group, a carbazolyl group, or a phenanthrolinyl group, each unsubstituted or substituted with deuterium, a halogen, a hydroxyl group, a carboxylate group, an ester moiety, a C₁-C₂₀ alkyl group, a C₁-C₂₀ alkoxy group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyridinyl group, a pyrazinyl group, a pyrimidinyl group, a pyridazinyl group, a quinolinyl group, an isoquinolinyl group, a benzoquinolinyl group, a quinoxalinyl group, a quinazolinyl group, a cinnolinyl group, a carbazolyl group, a phenanthrolinyl group, or any combination thereof.

In more detail, in Formula 1, R₁₃ and R₁₄ may each independently be a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, or a chrysenyl group, each unsubstituted or substituted with deuterium, a halogen, a hydroxyl group, a carboxylate group, ab ester moiety, a C₁-C₂₀ alkyl group, a C₁-C₂₀ alkoxy group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, and/or a combination thereof.

In at least one embodiment, the amine compound may be represented by one of Formulae 1-1 to 1-4:

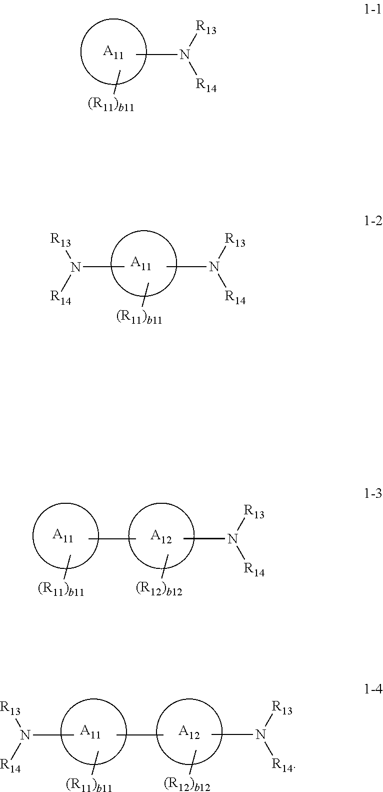

In Formulae 1-1 to 1-4,
A₁₁, A₁₂, R₁₁ to R₁₄, b11, and b12 are each referred to the definitions of those in Formula 1.

In at least one embodiment, the amine compound may have a symmetric structure. For example, the amine compound may be represented by Formula 1-2 or 1-4 in which R₁₃(s) are identical to each other and R₁₄(s) may be identical to each other.

In at least one embodiment, the amine compound may be selected from Group I:

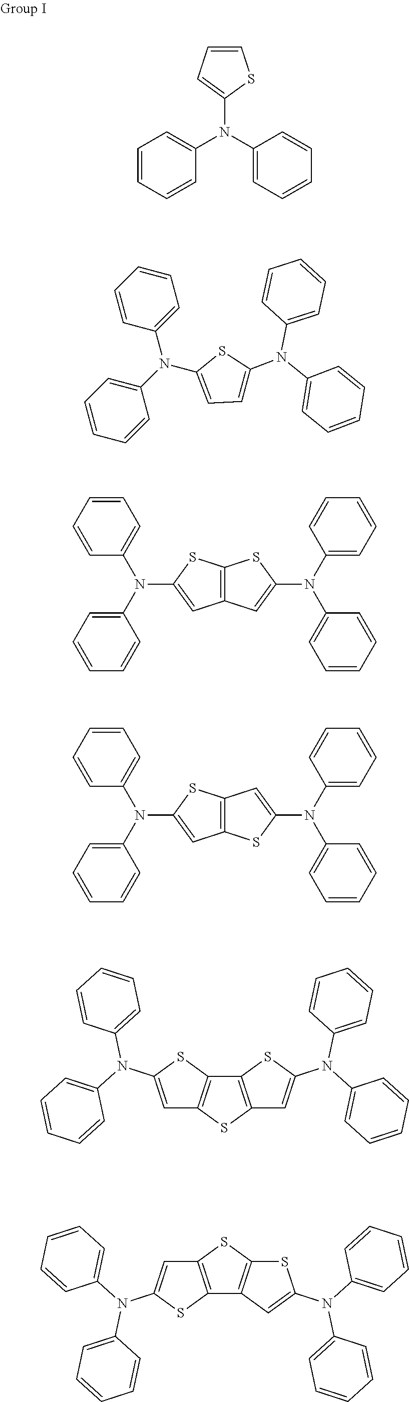 [SMILES: c1ccc(N(c2ccccc2)c2cccs2)cc1 ; c1ccc(N(c2ccccc2)c2ccc(N(c3ccccc3)c3ccccc3)s2)cc1 ; c1ccc(N(c2ccccc2)c2cc3cc(N(c4ccccc4)c4ccccc4)sc3s2)cc1 ; c1ccc(N(c2ccccc2)c2cc3sc(N(c4ccccc4)c4ccccc4)cc3s2)cc1 ; c1ccc(N(c2ccccc2)c2cc3sc4cc(N(c5ccccc5)c5ccccc5)sc4c3s2)cc1 ; c1ccc(N(c2ccccc2)c2cc3c(s2)sc2cc(N(c4ccccc4)c4ccccc4)sc23)cc1]

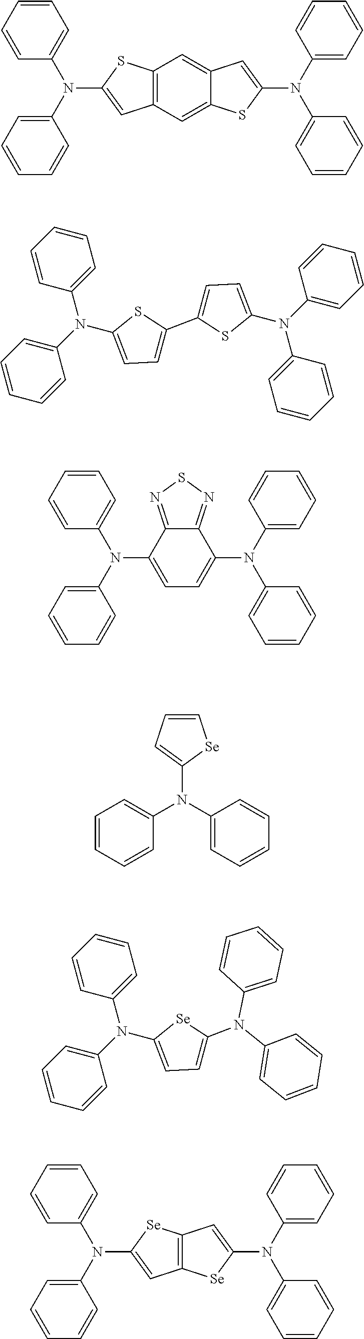 [SMILES: c1ccc(N(c2ccccc2)c2cc3cc4sc(N(c5ccccc5)c5ccccc5)cc4cc3s2)cc1 ; c1ccc(N(c2ccccc2)c2ccc(-c3ccc(N(c4ccccc4)c4ccccc4)s3)s2)cc1 ; c1ccc(N(c2ccccc2)c2ccc(N(c3ccccc3)c3ccccc3)c3nsnc23)cc1 ; c1ccc(N(c2ccccc2)c2ccc[se]2)cc1 ; c1ccc(N(c2ccccc2)c2ccc(N(c3ccccc3)c3ccccc3)[se]2)cc1 ; c1ccc(N(c2ccccc2)c2cc3[se]c(N(c4ccccc4)c4ccccc4)cc3[se]2)cc1]

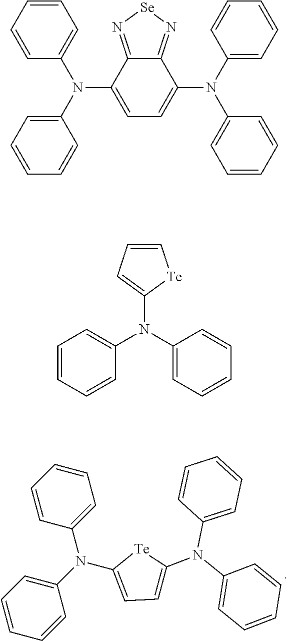

Gaussian simulation according to density functional theory (DFT) was performed on triphenylamine and some of the amine compounds above, so as to calculate highest occupied molecular orbital (HOMO) energy level, lowest unoccupied molecular orbital (LUMO) energy level, and ΔE (eV)=HOMO−LUMO. The results are shown in Table 1.

TABLE 1

| | | HOMO | LUMO | ΔE |
|---|---|---|---|---|
| # | Structure | (eV) | (eV) | (eV) |
| 1 | 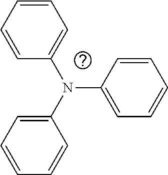 [SMILES: c1ccc(N(c2ccccc2)c2ccccc2)cc1] | −5.13 | −0.59 | 4.54 |
| 2 | 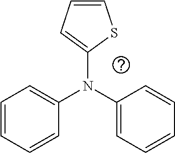 [SMILES: c1ccc(N(c2ccccc2)c2cccs2)cc1] | −5.19 | −0.69 | 4.51 |
| 3 | 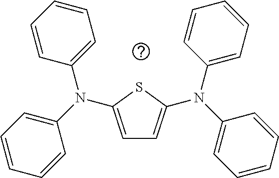 [SMILES: c1ccc(N(c2ccccc2)c2ccc(N(c3ccccc3)c3ccccc3)s2)cc1] | −5.16 | −0.80 | 4.36 |
| 4 | 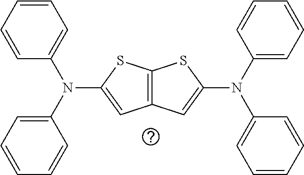 [SMILES: c1ccc(N(c2ccccc2)c2cc3cc(N(c4ccccc4)c4ccccc4)sc3s2)cc1] | −5.06 | −0.82 | 4.24 |
| 5 | 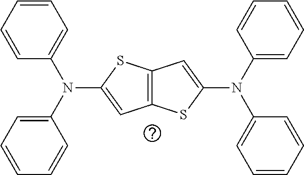 [SMILES: c1ccc(N(c2ccccc2)c2cc3sc(N(c4ccccc4)c4ccccc4)cc3s2)cc1] | −5.10 | −1.00 | 4.11 |
| 6 | 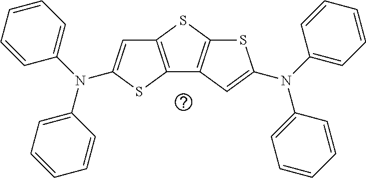 [SMILES: c1ccc(N(c2ccccc2)c2cc3c(s2)sc2cc(N(c4ccccc4)c4ccccc4)sc23)cc1] | −4.92 | −1.13 | 3.79 |
| 7 | 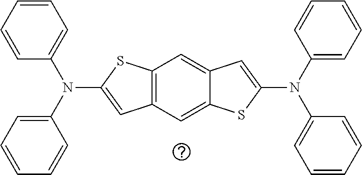 [SMILES: c1ccc(N(c2ccccc2)c2cc3cc4sc(N(c5ccccc5)c5ccccc5)cc4cc3s2)cc1] | −4.80 | −1.16 | 3.64 |
| 8 | 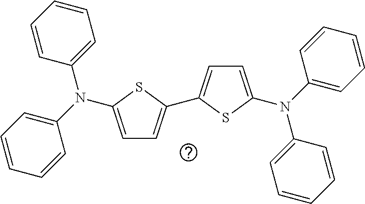 [SMILES: c1ccc(N(c2ccccc2)c2ccc(-c3ccc(N(c4ccccc4)c4ccccc4)s3)s2)cc1] | −4.86 | −1.46 | 3.40 |
| | | HOMO | | |
| # | Structure | (eV) | LUMO | ΔE |
| 9 | 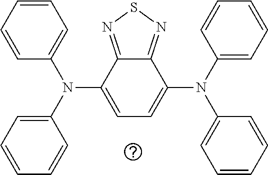 [SMILES: c1ccc(N(c2ccccc2)c2ccc(N(c3ccccc3)c3ccccc3)c3nsnc23)cc1] | −4.92 | −2.34 | 2.59 |
| 10 | 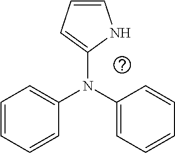 [SMILES: c1ccc(N(c2ccccc2)c2ccc[nH]2)cc1] | −4.97 | −0.86 | 4.10 |
| 11 | 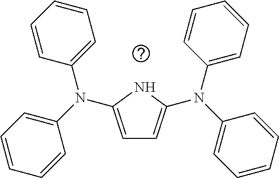 [SMILES: c1ccc(N(c2ccccc2)c2ccc(N(c3ccccc3)c3ccccc3)[nH]2)cc1] | −4.92 | −1.13 | 3.79 |
| 12 | 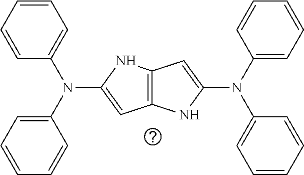 [SMILES: c1ccc(N(c2ccccc2)c2cc3[nH]c(N(c4ccccc4)c4ccccc4)cc3[nH]2)cc1] | −4.90 | −0.96 | 3.94 |
| 13 | 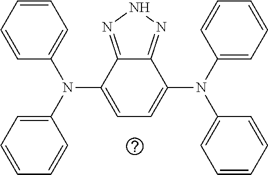 [SMILES: c1ccc(N(c2ccccc2)c2ccc(N(c3ccccc3)c3ccccc3)c3n[nH]nc23)cc1] | −4.90 | −2.46 | 2.44 |
| 14 | 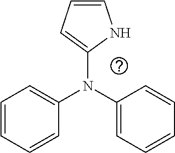 [SMILES: c1ccc(N(c2ccccc2)c2ccc[nH]2)cc1] | −4.85 | −1.23 | 3.62 |
| 15 | 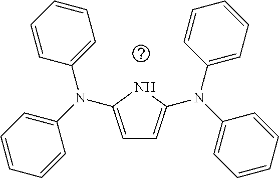 [SMILES: c1ccc(N(c2ccccc2)c2ccc(N(c3ccccc3)c3ccccc3)[nH]2)cc1] | −4.89 | −1.25 | 3.64 |

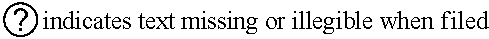

EUV (13.5 nm) has a shorter wavelength compared to, e.g., ArF immersion light sources (193 nm) and therefore can be utilized to produce patterns with finer patterns with smaller pitches. However, since EUV (13.5 nm) typically has a lower number of photons compared to the ArF immersion light sources, the lower the exposure dose is, the greater the noise at the boundary area between the area exposed by the EUV light source and the unexposed area that is not exposed. In other words, the noise in an EUV exposure is typically inversely proportional to the exposure dose such that the lower the exposure dose the greater the potential noise. In the case of a lithography process using an EUV light source, in order to compensate for this, a larger amount of a photoacid generator may be used compared to a lithography process using another light source having the same amount of light. However, when the resist composition includes a high content of a photoacid generator, the glass transition temperature (Tg) of the base resin may change and thermal stability thereof may deteriorate. In addition, the resolution of resist patterns formed due to a photoacid generator remaining during a lithography process using an EUV light source, may deteriorate.

The amine compound represented by Formula 1 may be configured to generate electrons by high-energy rays, especially EUV light sources, whereas the photoacid generator may not directly absorb EUV light sources. For example, the amine compound represented by Formula 1 may be ionized by high-energy rays to generate radical cations and electrons. Subsequently, with the electrons loss to some of the surrounding molecules, the resulting electrons may then react with a photoacid generator to produce acids from the photoacid generator. In particular, the amine compound represented by Formula 1 has a relatively high HOMO energy level and a small band gap, and thus a resist composition including the amine compound may be configured to generate an acid even at a relatively low amount of light, thereby improving resolution of resist patterns obtained by using the resist composition.

Also, since the amine compound represented by Formula 1 has suitable solubility in a solvent used in the resist composition, patterns of improved quality may be provided.

### [Resist Composition]

Another aspect of the disclosure provides a resist composition including the amine compound, a polymer, a photoacid generator, and the solvent. In at least one example, the solvent may be an organic solvent. The resist composition may have, for example, improved developability and/or improved resolution.

The solubility of the resist composition in a developer is changed by exposure to high energy ray. The resist composition may be a positive resist composition corresponding to a case where an exposed region of the resist film is dissolved and removed to form a positive resist pattern, or a negative resist composition corresponding to a case where an unexposed region of the resist film is dissolved and removed to form a negative resist pattern. In addition, the resist composition according to at least one embodiment may be used for an alkali developing process using an alkali developer for a developing process in forming a resist pattern, and/or may be used for a solvent developing process using a developer containing an organic solvent for the developing process (hereinafter referred to as an organic developer).

The amine compound may be included in an amount of about 0.1 parts by weight to about 10 parts by weight based on 100 parts by weight of the resist composition. In detail, the amine compound may be included in an amount of about 0.5 parts by weight to about 5 parts by weight based on 100 parts by weight of the resist composition. When the amount of the amine compound is satisfied within these ranges, the amine compound may exhibit functions as a sensitizer at an appropriate level, and formation of foreign particles may be reduced due to loss of any performance, e.g., a decrease in sensitivity and/or a lack of solubility.

Regarding the amine compound as described above, the polymer, the photoacid generator, the organic solvent, and optional components such as a quencher contained as necessary will be described below.

### <Polymer>

The polymer may include at least one of a first repeating unit represented by Formula 2 and a second repeating unit represented by Formula 3:

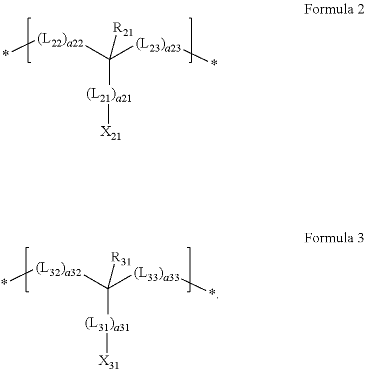

In Formulae 2 and 3,

L₂₁ to L₂₃ and L₃₁ to L₃₃ may each independently be: a single bond; O; S; C(═O); C(═O)O; OC(═O); C(═O)NH; NHC(═O); S(═O); S(═O)₂O; OS(═O)₂; and/or a linear, branched, or cyclic C₁-C₃₀ divalent hydrocarbon group that optionally includes a heteroatom,
a21 to a23 and a31 to a33 may each independently be an integer from 1 to 4,
R₂₁ and R₃₁ may each independently be: hydrogen; deuterium; a halogen; a cyano group; a hydroxyl group; an amino group; a carboxylate group; a thiol group; a carbonyl moiety; an ester moiety; a sulfonate moiety; a carbonate moiety; a lactone moiety; a sultone moiety; a carboxylic anhydride moiety; or a linear, branched, or cyclic C₁-C₃₀ monovalent hydrocarbon group that optionally includes a heteroatom,
X₂₁ may be an acid labile group,
X₃₁ may be a non-acid labile group, and
* indicates a binding site to a neighboring atom.

In one or more embodiments, in Formulae 2 and 3, L₂₁ to L₂₃ and L₃₁ to L₃₃ may each independently be: a single bond; O; C(═O); C(═O)O; OC(═O); C(═O)NH; NHC(═O); S(═O); S(═O)₂O; OS(═O)₂; and a C₁-C₂₀ alkylene group, a C₃-C₂₀ cycloalkylene group, a C₃-C₂₀ heterocycloalkylene group, a C₂-C₂₀ alkenylene group, a C₃-C₂₀ cycloalkenylene group, a C₃-C₂₀ heterocycloalkenylene group, a C₆-C₂₀ arylene group, and a C₁-C₂₀ heteroarylene group, each unsubstituted or substituted with deuterium, a halogen, a cyano group, a hydroxyl group, an amino group, a carboxylate group, a thiol group, a carbonyl moiety, an ester moiety, a sulfonate moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, and/or a combination thereof.

In one or more embodiments, in Formulae 2 and 3, L₂₁ to L₂₃ and L₃₁ to L₃₃ may each independently be: a single bond; O; C(═O); C(═O)O; OC(═O); C(═O)NH; NHC(═O); S(═O); S(═O)₂O; OS(═O)₂; and a C₁-C₂₀ alkylene group, a C₃-C₂₀ cycloalkylene group, a C₃-C₂₀ heterocycloalkylene group, a phenylene group, and a naphthylene group, each unsubstituted or substituted with deuterium, a halogen, a C₁-C₂₀ alkyl group, a C₁-C₂₀ halogenated alkyl group, a C₁-C₂₀ alkoxy group, a phenyl group, a naphthyl group, and/or a combination thereof.

In Formulae 2 and 3, a21 to a23 and a31 to a33 indicate the number of repetitions of L₂₁ to L₂₃ and the number of repetitions of L₃₁ to L₃₃, respectively.

In one or more embodiments, in Formulae 2 and 3, a21 to a23 and a31 to a33 may each independently be an integer from 1 to 3.

In one or more embodiments, in Formulae 2 and 3, a21 to a23 and a31 to a33 may each independently be 1.

In one or more embodiments, in Formulae 2 and 3, R₂₁ and R₃₁ may each independently be: hydrogen; deuterium; a halogen; a cyano group; a hydroxyl group; an amino group; a carboxylate group; a thiol group; and a C₁-C₂₀ alkyl group, a C₃-C₂₀ cycloalkyl group, and a C₆-C₂₀ aryl group, each unsubstituted or substituted with deuterium, a halogen, a cyano group, a hydroxyl group, an amino group, a carboxylate group, a thiol group, a carbonyl moiety, an ester moiety, a sulfonate moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, and/or a combination thereof.

In one or more embodiments, in Formulae 2 and 3, R₂₁ and R₃₁ may each independently be: hydrogen; deuterium; a halogen; a cyano group; a hydroxyl group; an amino group; a carboxylate group; a thiol group; a C₁-C₂₀ alkyl group; a C₁-C₂₀ halogenated alkyl group; a C₃-C₂₀ cycloalkyl group; or a C₆-C₂₀ aryl group.

In one or more embodiments, in Formula 2, X₂₁ may be represented by one of Formulae 6-1 to 6-12:

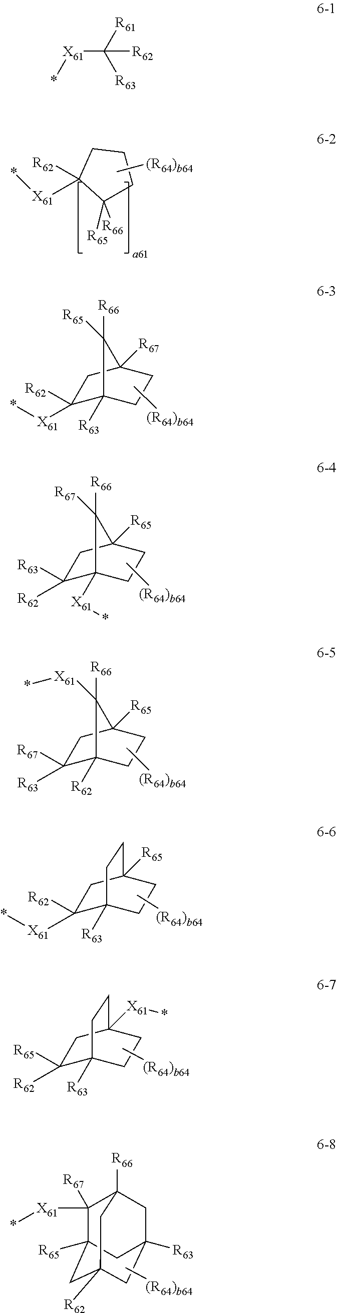

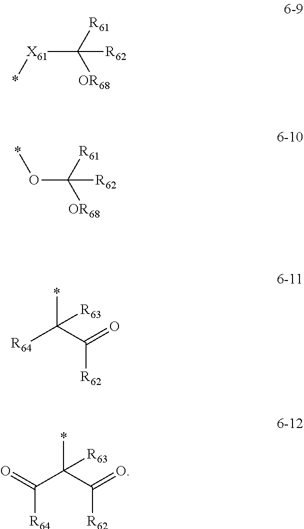

In Formulae 6-1 to 6-12,
X₆₁ may be an ester moiety, a carbonate moiety, a carbamate moiety, or a sulfonate moiety,
a61 may be an integer from 0 to 6,
R₆₁ and R₆₈ may each independently be a linear, branched or cyclic C₁-C₂₀ monovalent hydrocarbon group that optionally includes a heteroatom,
R₆₂ to R₆₇ may each independently be: hydrogen; deuterium; a halogen; a cyano group; a hydroxyl group; an amino group; a carboxylate group; a thiol group; a carbonyl moiety; an ester moiety; a sulfonate moiety; a carbonate moiety; a carbamate moiety; a lactone moiety; a sultone moiety; a carboxylic anhydride moiety; or a linear, branched, or cyclic C₁-C₃₀ monovalent hydrocarbon group that optionally includes a heteroatom,
two adjacent groups of R₆₁ to R₆₈ may optionally be bonded to each other to form a ring,
b64 may be an integer from 1 to 10, and
* indicates a binding site to a neighboring atom.

In particular, X₂₁ in Formula 2 may be represented by one of Formulae 6-1 and 6-3.

In at least one embodiment, the first repeating unit represented by Formula 2 may be represented by one of Formulae 2-1 and 2-2:

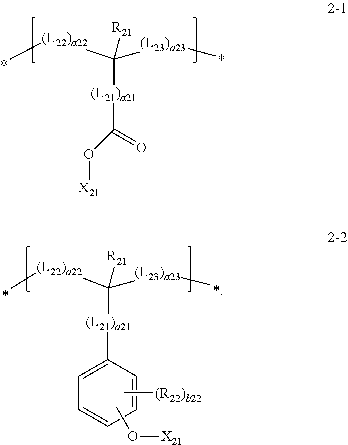

In Formulae 2-1 and 2-2,
L₂₁ to L₂₃, a21 to a23, R₂₁, and X₂₁ are the same as described herein,
R₂₂ may be: hydrogen; or a linear, branched, and/or cyclic C₁-C₃₀ monovalent group that optionally includes a heteroatom,
b22 may be an integer from 1 to 4, and
* indicates a binding site to a neighboring atom.

In one or more embodiments, the first repeating unit may be selected from Group II:

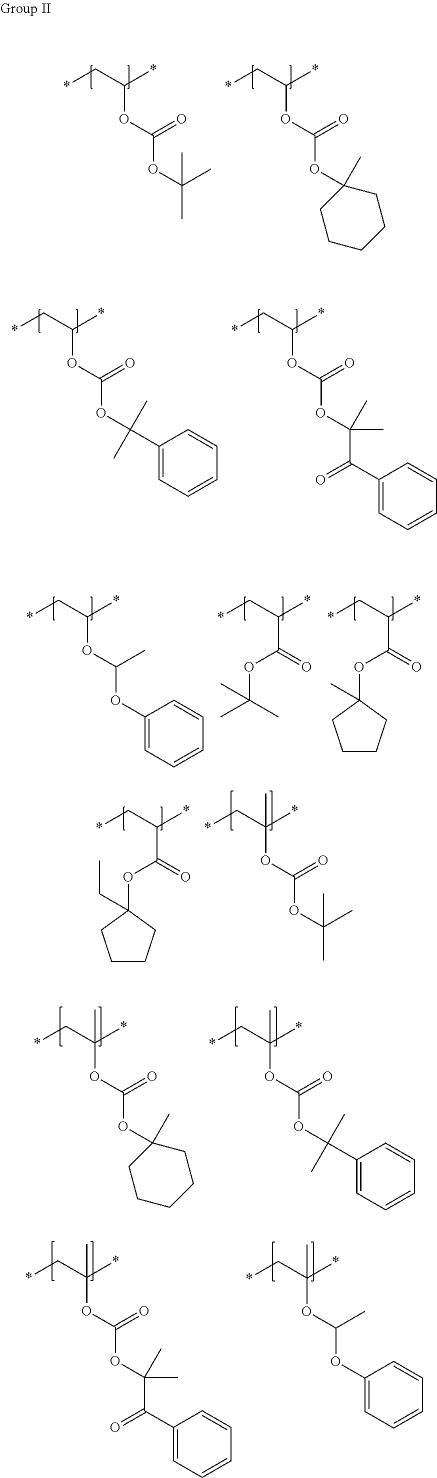

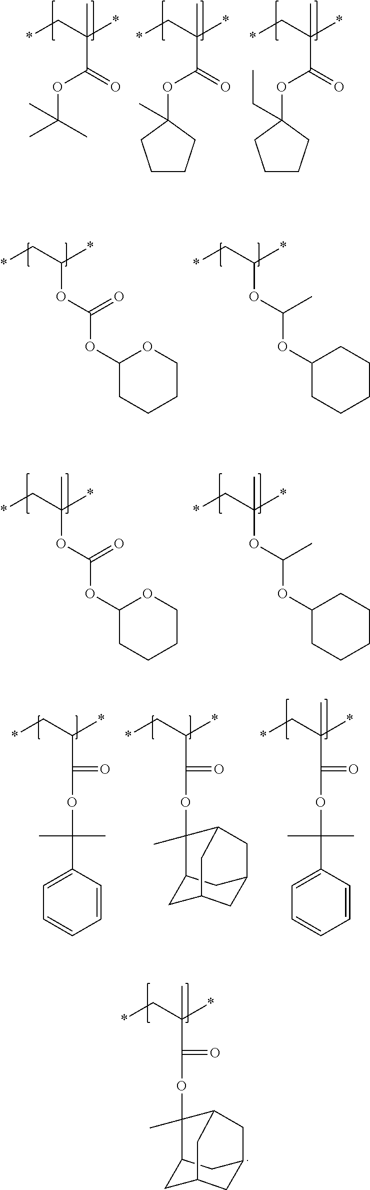

In at least one embodiment, in Formula 3, X₃₁ may be: hydrogen; a halogen; a cyano group; a hydroxyl group; a carboxylate group; a thiol group; an amino group; or a linear, branched, and/or cyclic C₁-C₃₀ monovalent hydrocarbon group that optionally includes at least one polar moiety selected from a halogen, a cyano group, a hydroxyl group, a thiol group, a carboxylate group, O, C═O, C(═O)O, OC(═O), S(═O)₂O, OS(═O)₂, a lactone moiety, a sultone moiety, and a carboxylic anhydride moiety.

In one or more embodiments, in Formula 3, X₃₁ may be hydrogen or a hydroxyl group, or may be represented by one of Formulae 5-1 to 5-12:

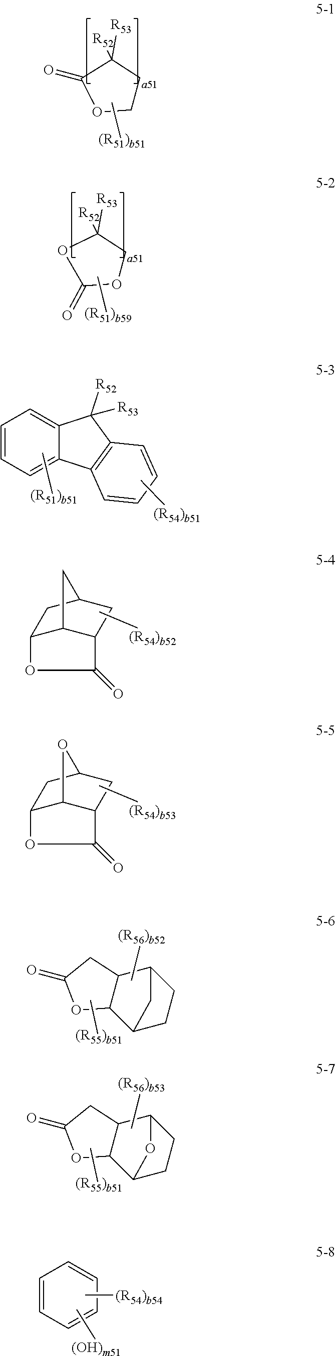

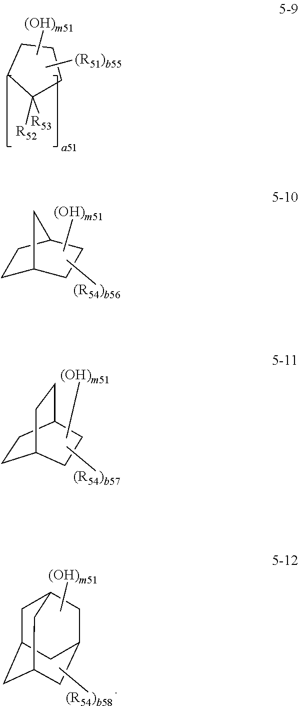

In Formulae 5-1 to 5-12,
a51 maybe 1 or 2,

R₅₁ to R₅₆ may each independently be: a binding site to a neighboring atom; hydrogen; a
hydroxyl group; a carboxylate group; a carbonyl moiety, an ester moiety; a carbonate moiety; a carbamate moiety; a lactone moiety; a carboxylic anhydride moiety; and/or a linear, branched, or cyclic C₁-C₂₀ monovalent hydrocarbon group,
one of R₅₁ to R₅₃, one of R₅₄(s), and one of R₅₅ and R₅₆ each indicate a binding site to a neighboring atom,
b51 may be an integer from 1 to 4,
b52 may be an integer from 1 to 10,
b53 may be an integer from 1 to 8,
b54 may be an integer from 1 to 5,
b55 may be an integer from 1 to 7,
b56 may be an integer from 1 to 11,
b57 may be an integer from 1 to 13,
b58 may be an integer from 1 to 15,
b59 may be 1 or 2, and
m51 may be an integer from 1 to 4.

In particular, X₃₁ in Formula 3 may be selected from a hydroxyl group and a group represented by one of Formulae 5-1, 5-6, 5-8, and 5-10.

In at least one embodiment, the second repeating unit represented by Formula 3 may be represented by one of Formulae 3-1 and 3-2:

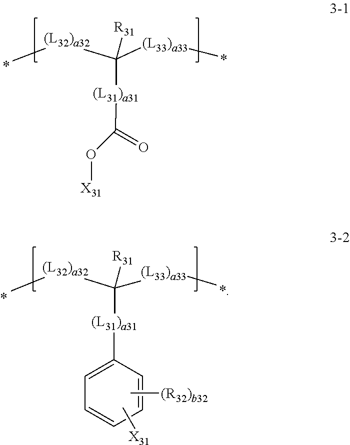

In Formulae 3-1 and 3-2,
L₃₁ to L₃₃, a31 to a33, R₃₁, and X₃₁ are the same as described herein,
R₃₂ may be: hydrogen; or a linear, branched, or cyclic C₁-C₃₀ monovalent group that optionally includes a heteroatom,
b32 may be an integer from 1 to 4, and
* indicates a binding site to a neighboring atom.

In one or more embodiments, the second repeating unit may be selected from Group III:

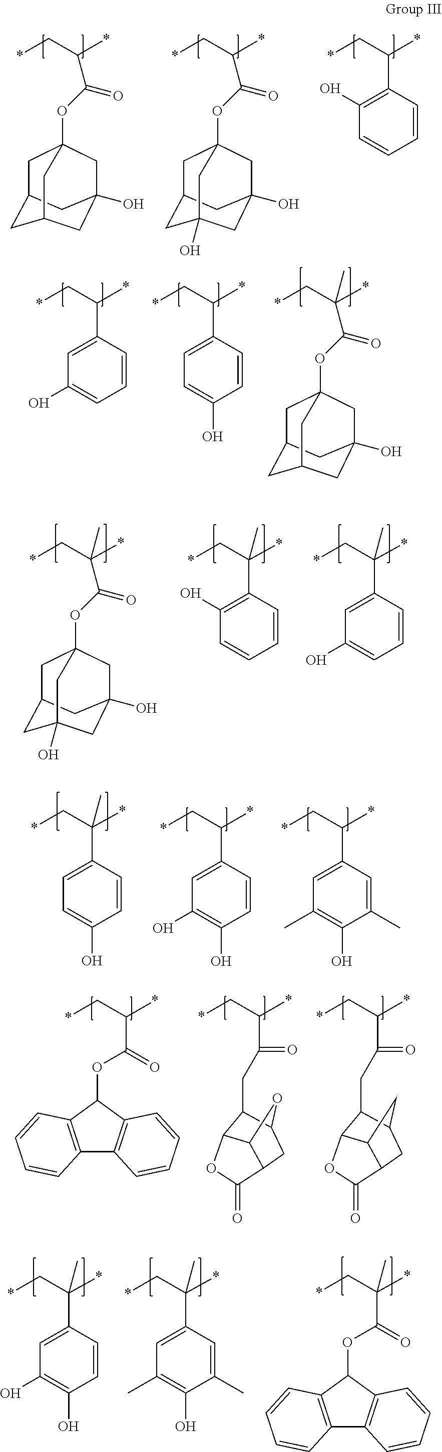

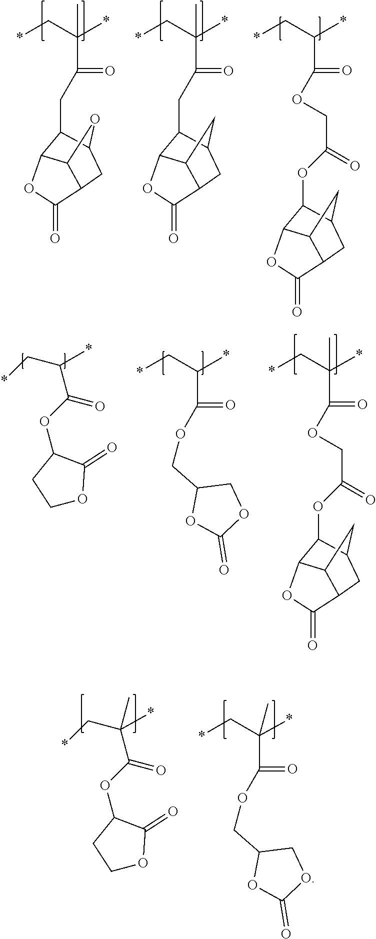

In at least one embodiment, the polymer may include the first repeating unit in an amount in a range of about 1 mol % to about 100 mol %, about 5 mol % to about 100 mol %, and about 10 mol % to about 100 mol %.

In one or more embodiments, the polymer may not include the second repeating unit (e.g., consist of the first repeating unit).

In one or more embodiments, the polymer may include the second repeating unit in an amount in a range of about 0 mol % to about 99 mol %, about 1 mol % to about 99 mol %, and about 10 mol % to about 90 mol %.

In one or more embodiments, the polymer may consist of the first repeating unit and the second repeating unit. In one or more embodiments, the polymer may include the first repeating unit in an amount in a range of about 1 mol % to about 99 mol % or about 10 mol % to about 90 mol %, and the second repeating unit in an amount in a range of about 1 mol % to about 99 mol % or about 10 mol % to about 90 mol %.

The polymer may have a weight average molecular weight (Mw) in a range of about 1,000 to 500,000, for example, about 3,000 to about 100,000 or about 5,000 to about 50,000, wherein the weight average molecular weight is measured by gel permeation chromatography using a tetrahydrofuran solvent and polystyrene as a standard material.

The polymer may have polydispersity index (PDI: Mw/Mn) in a range of about 1.0 to about 3.0, for example, about 1.0 to about 2.5. Within these ranges, the possibility of foreign substances remaining on the pattern may be lowered, or deterioration of a pattern profile may be minimized. Accordingly, the resist composition including the polymer may be more suitable for forming fine patterns.

One type of the polymer may be used, or a combination of two or more different types of the polymer may be used.

### <Photoacid Generator>

The photoacid generator may be any compound capable of generating an acid when exposed to high energy rays such as UV, deep UV DUV, electron beam (EB), EUV, X-rays, α-rays, γ-rays, and/or the like.

The photoacid generator may include a sulfonium salt, an iodonium salt, or a combination thereof.

In at least one embodiment, the photoacid generator may be represented by Formula 7:

B₇₁⁺A₇₁⁻ Formula 7

In Formula 7,

B₇₁⁺ may be represented by Formula 7A, A₇₁⁻ may be represented by one of Formulae 7B to 7D, and
B₇₁⁺ and A₇₁⁻ may optionally be linked to each other via a carbon-carbon covalent bond:

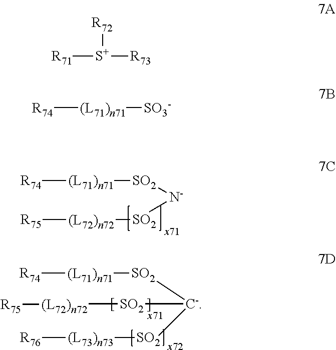

In Formulae 7A to 7D,
L₇₁ to L₇₃ may each independently be a single bond or CRR′,
R and R′ may each independently be hydrogen, deuterium, a halogen, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group, or a C₃-C₃₀ cycloalkoxy group,
n71 to n73 may each independently be 1, 2, or 3,
x71 and x72 may each independently be 0 or 1,
R₇₁ to R₇₃ may each independently be a linear, branched, or cyclic C₁-C₃₀ monovalent hydrocarbon group that optionally includes a heteroatom,
two adjacent groups among R₇₁ to R₇₃ may optionally be bonded to each other to form a condensed ring, and
R₇₄ to R₇₆ may each independently be: hydrogen; a halogen; or a linear, branched, or cyclic C₁-C₃₀ monovalent hydrocarbon group that optionally includes a heteroatom.

For example, in Formula 7, B₇₁⁺ may be represented by Formula 7A, and A₇₁⁻ may be represented by Formula 7B. In detail, each of R₇₁ to R₇₃ in Formula 7 may be a phenyl group.

The photoacid generator may be included in an amount in a range of about 0.01 parts by weight to about 40 parts by weight, about 0.1 parts by weight to about 40 parts by weight, or about 0.1 parts by weight to about 20 parts by weight, based on 100 parts by weight of the polymer. When the amount of the photodegradable quencher is satisfied within the ranges above, proper resolution may be achieved, and problems related to foreign particles after development or during stripping may be reduced.

In at one embodiment, one type of the photoacid generator may be used, and/or a combination of two or more different types of the photoacid generator may be used.

### <Organic Solvent>

The organic solvent included in the resist composition may be selected from a solvent capable of dissolving and/or dispersing the amine compound, the polymer, the photoacid generator, and optional components (such as a quencher contained as necessary). In at least some embodiments, one type of organic solvent may be used, and/or a combination of two or more different types of organic solvent may be used. In addition, a mixed solvent in which water and the organic solvent are mixed may be used.

Examples of the organic solvent may include an alcohol-based solvent, an ether-based solvent, a ketone-based solvent, an amide-based solvent, an ester-based solvent, a sulfoxide-based solvent, a hydrocarbon-based solvent, and the like.

Examples of the alcohol-based solvent may include: a monoalcohol-based solvent, such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxybutanol, n-hexanol, 2-methylpentanol, sec-hexanol, 2-ethylbutanol, 4-methyl-2-pentanol (MIBC), sec-heptanol, 3-heptanol, n-octanol, 2-ethylhexanol, sec-octanol, n-nonylalcohol, 2,6-dimethyl-4-heptanol, n-decanol, sec-undecyl alcohol, trimethylnonyl alcohol, sec-tetradecyl alcohol, sec-heptadecyl alcohol, furfuryl alcohol, phenol, cyclohexanol, methylcyclohexane alcohol, 3,3,5-trimethylcyclohexanol, benzyl alcohol, diacetone alcohol, and the like; a polyhydric alcohol solvent, such as ethylene glycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethylene glycol, dipropylene glycol, triethylene glycol, and tripropylene glycol; and polyhydric alcohol-containing ether solvents such as ethylene glycol monomethyl ether, ethylene glycol monoethyl ether, ethylene glycol monopropyl ether, ethylene glycol monobutyl ether, ethylene glycol monohexyl ether, ethylene glycol monophenyl ether, ethylene glycol mono-2-ethylbutyl ether, diethylene glycol monomethyl ether, diethylene glycol monoethyl ether, diethylene glycol monopropyl ether, diethylene glycol monobutyl ether, diethylene glycol monohexyl ether, diethylene glycol dimethyl ether, propylene glycol monomethyl ether, propylene glycol dimethyl ether, propylene glycol monoethyl ether, propylene glycol monopropyl ether, propylene glycol monobutyl ether, dipropylene glycol monomethyl ether, dipropylene glycol monoethyl ether, dipropylene glycol monopropyl ether, and/or the like.

Examples of the ether-based solvent may include: a dialkyl ether-based solvent (such as diethyl ether, dipropyl ether, dibutyl ether, and/or the like); a cyclic ether-based solvent (such as tetrahydrofuran, tetrahydropyran, and the like); an aromatic ring-containing ether-based solvent (such as diphenyl ether, anisole, and/or the like); and/or the like.

Examples of the ketone-based solvent may include: a chain ketone solvent (such as acetone, methylethylketone, methyl-n-propyl ketone, methyl-n-butyl ketone, methyl-n-pentyl ketone, diethyl ketone, methyl isobutyl ketone, 2-heptanone, ethyl-n-butyl ketone, methyl-n-hexy ketone, diisobutyl ketone, trimethyl nonanone, and/or the like); a cyclic ketone-based solvent (such as cyclopentanone, cyclohexanone, cycloheptanone, cyclooctanone, methylcyclohexanone, and/or the like); 2,4-pentandione; acetonyl acetone; acetphenone; and/or the like.

Examples of the amide-based solvent may include: a cyclic amide-based solvent (such as N,N′-dimethylimidazolidinone, N-methyl-2-pyrrolidone, and/or the like); a chain amide-based solvent (such as N-methylformamide, N,N-dimethylformamide, N,N-diethylformamide, acetamide, N-methylacetamide, N,N-dimethylacetamide, N-methylpropionamide, and/or the like); and/or the like.

Examples of ester solvent may include: an acetate ester solvent (such as methyl acetate, ethyl acetate, n-propyl acetate, isopropyl acetate, n-butyl acetate, isobutyl acetate, sec-butyl acetate, T-butyl acetate, n-pentyl acetate, isopentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, n-nonyl acetate, and/or the like); a polyhydric alcohol-containing ether carboxylate solvent (such as ethylene glycol monomethyl ether acetate, ethylene glycol monoethyl ether acetate, diethylene glycol monomethyl ether acetate, diethylene glycol monoethyl ether acetate, diethylene glycol mono-n-butyl ether acetate, propylene glycol monomethyl ether acetate, propylene glycol monoethyl ether acetate, propylene glycol monopropyl ether acetate, propylene glycol monobutyl ether acetate, dipropylene glycol monomethyl ether acetate, dipropylene glycol monoethyl ether acetate, and/or the like); a lactone solvent (such as γ-butyrolactone and δ-valerolactone; carbonate solvents such as dimethyl carbonate, diethyl carbonate, ethylene carbonate, and propylene carbonate; lactate ester solvents such as methyl lactate, ethyl lactate, n-butyl lactate, n-amyl lactate, and/or the like); glycoldiacetate; methoxytriglycol acetate; ethyl propionate; n-butyl propionate; isoamyl propionate; diethyloxalate; di-n-butyloxalate; methyl acetoacetate; ethyl acetoacetate; diethyl malonate; dimethyl phthalate; diethyl phthalate; and/or the like.

Examples of the sulfoxide-based solvent may include dimethyl sulfoxide, diethyl sulfoxide, and/or the like.

Examples of the hydrocarbon-based solvent may include: an aliphatic hydrocarbon-based solvent, such as n-pentane, isopentane, n-hexane, isohexane, n-heptane, isoheptane, 2,2,4-trimethyl pentane, n-octane, isooctane, cyclohexane, methylcyclohexane, and the like; an aromatic hydrocarbon-based solvent (such as benzene, toluene, xylene, mesitylene, ethyl benzene, trimethyl benzene, methylethyl benzene, n-propyl benzene, isopropyl benzene, diethyl benzene, isobutyl benzene, triethyl benzene, diisopropyl benzene, n-amylnaphthalene, and/or the like); and/or the like.

In at least one embodiment, the organic solvent may be selected from an alcohol-based solvent, an amide-based solvent, an ester-based solvent, a sulfoxide-based solvent, and any combination thereof. In one or more embodiments, the organic solvent may be selected from propylene glycol monomethyl ether, propylene glycol monoethyl ether, propylene glycol monomethyl ether acetate, N-methyl-2-pyrrolidone, N,N-dimethylacetamide, ethyl lactate, dimethyl sulfoxide, and/or a combination thereof.

In at least one embodiment, when an acetal-type acid labile group is used, the organic solvent may further include alcohol having a high boiling point, such as diethylene glycol, propylene glycol, glycerol, 1,4-butanediol, or 1,3-butanediol, to accelerate a deprotection reaction of acetal.

The organic solvent may be used in an amount in a range of about 200 parts by weight to about 20,000 parts by weight, for example, about 2,000 to about 10,000 parts by weight, based on 100 parts by weight of the polymer.

### <Quencher>

The resist composition may further include a quencher.

The quencher may be a salt that generates an acid with weaker acidity than the acid generated from the photoacid generator.

The quencher may include an ammonium salt, a sulfonium salt, an iodonium salt, and/or a combination thereof.

In at least one embodiment, the quencher may be represented by Formula 8:

B₈₁⁺A₈₁⁻ Formula 8

In Formula 8,
B81⁺ may be represented by one of Formulae 8A to 8C, and A₈₁⁻ may be represented by one of Formulae 8D to 8F, and
B₈₁⁺ and A₈₁⁻ may optionally be linked to each other via a carbon-carbon covalent bond:

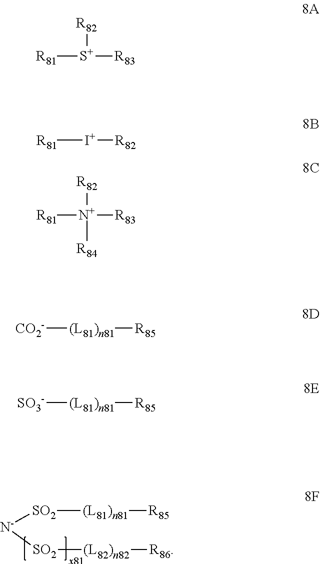

In Formulae 8A to 8F,
L₈₁ and L₈₂ may each independently be a single bond or CRR′,
R and R′ may each independently be hydrogen, deuterium, a halogen, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group, or a C₃-C₃₀ cycloalkoxy group,
n81 and n82 may each independently be 1, 2, or 3,
x81 may be 0 or 1,
R₈₁ to R₈₄ may each independently be a linear, branched, or cyclic C₁-C₃₀ monovalent hydrocarbon group that optionally includes a heteroatom,
two adjacent groups among R₈₁ to R₈₄ may optionally be bonded to each other to form a condensed ring, and
R₈₅ and R₈₆ may each independently be: hydrogen; a halogen; or a linear, branched, or cyclic C₁-C₃₀ monovalent hydrocarbon group that optionally includes a heteroatom.

The quencher may be included in an amount in a range of about 0 parts by weight to about 10 parts by weight, 0.05 parts by weight to about 5 parts by weight, or about 0.1 parts by weight to about 3 parts by weight, based on 100 parts by weight of the polymer. When the amount of the photodegradable quencher is satisfied within the ranges above, proper resolution may be achieved, and problems related to foreign particles after development or during stripping may be reduced.

One type of the surfactant may be used, or a combination of two or more different types of the surfactant may be used.

### <Optional Components>

The resist composition may further include a surfactant, a cross-linking agent, a leveling agent, a colorant, or any combination thereof, as needed.

The resist composition may further include a surfactant to improve a coating property and developability. Examples of the surfactant may include: a non-ionic surfactant, such as polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate, polyethylene glycol distearate, and the like; and/or the like. For use as the surfactant, a commercially available product may be used, and/or a synthetic product may be used. Examples of the commercially available surfactant may include KP341 (manufactured by Shin-Etsu Chemical Co., Ltd.), POLYFLOW No.75 and POLYFLOW No.95 (manufactured by Kyoeisha Chemical Co., Ltd.), FTOP EF301, FTOP EF303, and FTOP EF352 (manufactured by Mitsubishi Materials Electronic Chemicals Co., Ltd.), MEGAFACE F171 (registered trademark), MEGAFACE F173, R40, R41, and R43 (manufactured by DIC Corporation), Fluorad FC430 (registered trademark) and Fluorad FC431 (manufactured by 3M Company), AsahiGuard AG710 (product of AGC Corporation), Surflon S-382 (registered trademark), Surflon SC-101, Surflon SC-102, Surflon SC-103, Surflon SC-104, Surflon SC-105, and Surflon SC-106 (manufactured by AGC Seimi Chemical Co., Ltd.), and/or the like.

The surfactant may be included in an amount in a range of about 0 parts by weight to about 20 parts by weight based on 100 parts by weight of the polymer.

One type of the surfactant may be used, and/or a combination of two or more different types of the surfactant may be used.

A method of preparing the resist composition is not particularly limited, and for example, a method of mixing an amine compound, a polymer, a photoacid generator, and optional components added as necessary in an organic solvent may be used. The temperature or time at the time of mixing is not particularly limited. Following the mixing, filtration may be performed as necessary.

### [Pattern Formation Method]

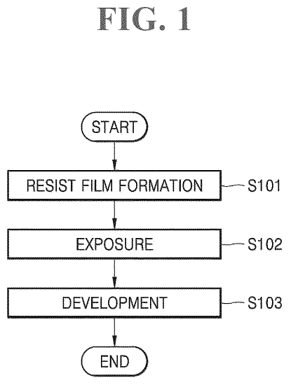

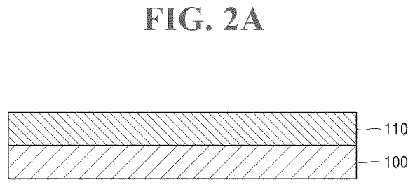

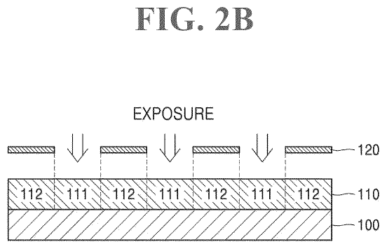

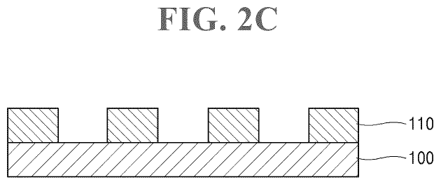

Hereinafter, a pattern formation method according to embodiments will be described in more detail with reference to FIGS. 1 and 2A to 2C. FIG. 1 is a flowchart representing a pattern forming method according to at least one embodiment, and FIG. 2 is a side cross-sectional view illustrating a pattern forming method according to at least one embodiment. Hereinafter, a pattern formation method utilizing a positive resist composition will be described in detail as an example, but embodiments are not limited thereto.

Referring to FIG. 1, the pattern formation method may include: forming a resist film by applying a resist composition (S101); exposing at least a portion of the resist film with high energy rays (S102); and developing the exposed resist film by using a developer (S103). The operations above may be repeated and/or omitted as necessary, and/or may be performed in different orders.

First, a substrate 100 is prepared. The substrate 100 may be, for example, a semiconductor substrate, such as a silicon substrate or a germanium substrate, or may be formed of glass, quartz, ceramic, copper, and the like. In at least one embodiment, the substrate 100 may include a Group III-V compound, such as GaP, GaAs, GaSb, and the like.

Referring to FIG. 2A, a resist film 110 may be formed by applying a resist composition onto the substrate 100 to a desired thickness by a coating method. As needed, the method may include heating (pre-baking (PB) or post-annealing baking (PAB) operations) to remove the organic solvent remaining in the resist film 110.

The coating method may include spin coating, dipping, roller coating, or other common coating methods. Among these methods, spin coating may be particularly used, and the resist film 110 having a desired thickness may be formed by adjusting the viscosity, concentration, and/or spinning speed of the resist composition. In at least one embodiment, the thickness of the resist film 110 may be in a range of about 10 nm to about 300 nm. In one or more embodiments, the thickness of the resist film 110 may be in a range of about 30 nm to about 200 nm.

The lower limit of a PB temperature may be 60° C. or higher, for example, 80° C. or higher. In addition, the upper limit of a PB temperature may be 150° C. or lower, for example, 140° C. or lower. The lower limit of a PB time may be 5 seconds or more, for example, 10 seconds or more. The upper limit of a PB time may be 600 seconds or less, for example, 300 seconds or less.

Before coating the resist composition onto the substrate 100, a target film to be etched (not shown) may be further formed on the substrate 100. The film to be etched may refer to a layer on which an image is transferred from a resist pattern and converted into a certain pattern.

In at least one embodiment, the film to be etched may be formed to include, for example, an insulating material, such as silicon oxide, silicon nitride, silicon oxynitride, and/or the like. In one or more embodiments, the film to be etched may be formed to include a conductive material, such as metal, metal nitride, metal silicide, or metal silicide nitride. In one or more embodiments, the film to be etched may be formed to include a semiconductor material such as polysilicon.

In at least one embodiment, an anti-reflection film may be further formed on the substrate 100 to increase (and/or maximize) the efficiency of the resist film 110. The anti-reflection film may be an organic-based anti-reflection layer and/or an inorganic-based anti-reflection layer.

In at least one embodiment, a protective film may be further provided on the resist film 110 in order to reduce the influence of alkaline impurities included in the process. Further, in the case of immersion exposure, for example, a protective film for immersion may be provided on the resist film 110 to avoid direct contact between the immersion medium and the resist film 110.

Next, referring to FIG. 2B, at least a portion of the resist film 110 may be exposed to high energy ray. For example, high energy ray passing through a mask 120 may be irradiated to at least a portion of the resist film 110. As such, the resist film 110 may include exposed regions 111 and unexposed regions 112.

In the exposing, the amine compound may be ionized to generate radical cations and electrons.

The exposure may be carried out by irradiating high-energy ray through a mask having a predetermined pattern and by using liquid, such as water or the like, as a medium in some cases.

Examples of the high-energy rays may include: ultraviolet rays; deep ultraviolet (DUV) rays; extreme ultraviolet (EUV) rays, wavelength of 13.5 nm); electromagnetic waves, such as X-rays, γ-rays, and the like; electron beam (EB) rays; charged particle beams (such as α rays and/or the like); and/or the like. The irradiation of such high-energy rays may be collectively referred to as “exposure.”

For use as a light source of the exposure, various types of irradiation including irradiating laser beams in the ultraviolet region, such as KrF excimer laser (wavelength of 248 nm), ArF excimer laser (wavelength of 193 nm), and F₂ excimer laser (wavelength of 157 nm), irradiating harmonic laser beams in the far ultraviolet or vacuum ultraviolet region by a wavelength conversion method using laser beams from a solid-state laser source (e.g., YAG or semiconductor laser), irradiating electron beams or EUV rays, and/or the like may be used. Upon the exposure, the exposure may be performed through a mask corresponding to a desired pattern. However, when the light source of the exposure is EBs, the exposure may be performed by direct drawing without using a mask.

The integral dose of the high-energy rays may be 2,000 mJ/cm² or less, for example, 500 mJ/cm² or less, in the case of using EUV rays as the high-energy rays. In addition, in the case of using EBs as the high-energy rays, the integral dose of the high-energy rays may be 5,000 μC/cm² or less, for example, 1,000 μC/cm² or less.

In addition, post-exposure baking (PEB) may be performed after the exposure. The lower limit of a PEB temperature may be 50° C. or higher, for example, 80° C. or higher. The upper limit of the PEB temperature may be 180° C. or lower, for example, 130° C. or lower. The lower limit of a PEB time may be 5 seconds or more, for example, 10 seconds or more. The upper limit of the PEB time may be 600 seconds or less, for example, 300 seconds or less.

Next, referring to FIG. 2C, the exposed resist film 110 may be developed by using a developer. The exposed area 111 may be washed away by the developer, whereas the unexposed area 112 may remain without being washed away by the developer.

For use as the developer, an alkali developer, a developer containing an organic solvent (hereinafter also referred to as “organic developer”), and the like may be used. As a developing method, a dipping method, a puddle method, a spray method, a dynamic administration method, and the like may be used. The developing temperature may be, for example, 5° C. or more and 60° C. or less, and the developing time may be, for example, 5 seconds or more and 300 seconds or less.

The alkali developer may be, for example, an alkaline aqueous solution which dissolves at least one alkaline compound, such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo[5.4.0]-7-undecene (DBU), 1,5-diazabicyclo[4.3.0]-5-nonene, and the like. The alkaline developer may further include a surfactant.

The lower limit of the amount of the alkaline compound in the alkaline developer may be 0.1 mass % or more, for example, 0.5 mass % or more, and for example, 1 mass % or more. In addition, the upper limit of the amount of the alkaline compound in the alkaline developer may be 20 mass % or less, for example, 10 mass % or less, and for example, 5 mass % or less.

After the development, a resulting resist pattern may be washed with ultrapure water, and subsequently, the water remaining on the substrate 100 and the pattern may be removed.

As an organic solvent contained in the organic developer, for example, the same organic solvent as the organic solvent described in the <Organic solvent> of the [Resist composition] may be used.

The lower limit of the amount of the organic solvent in the organic developer may be 80 mass % or more, for example, 90 mass % or more, and for example, 95 mass % or more, and for example, 99 mass % or more.

The organic developer may also include a surfactant. In addition, the organic developer may include a trace amount of moisture. In addition, upon the development, the solvent may be substituted with a solvent of a different kind from the organic developer to stop the development.

The resist pattern after development may be further cleaned by using a washing solution. For use as the washing solution, ultrapure water, rinsing liquid, and the like may be used. The rinsing liquid is not particularly limited as long as it does not dissolve the resist pattern, and a solution containing a general organic solvent may be used. For example, the rinsing liquid may be an alcohol-based solvent or an ester-based solvent. After the washing, the rinsing liquid remaining on the substrate and the pattern may be removed. In addition, when ultrapure water is used, the water remaining on the substrate and the pattern may be removed.

In addition, the developer may be used either individually or in a combination of two or more types.

In at least some embodiments, after the resist pattern is formed as described above, a patterned interconnection substrate may be obtained. An etching method may be performed. For example, the etching method may include dry etching using plasma gas; wet etching using an alkali solution, a cupric chloride solution, a ferric chloride solution; and/or the like.

In at least some embodiments, after forming the resist pattern, plating may be performed. Although not particularly limited, a plating method may include, for example, copper plating, solder plating, nickel plating, gold plating, and/or the like.

The resist pattern remaining after etching may be peeled with an organic solvent. Although not particularly limited, examples of the organic solvent may include propylene glycol monomethyl ether acetate (PGMEA) propylene glycol monomethyl ether (PGME), ethyl lactate (EL), and the like. Although not particularly limited, examples of the exfoliation method may include an immersion method, a spray method, and the like. In addition, the interconnection substrate on which the resist pattern is formed may be a multi-layer interconnection substrate or may have small-diameter through-holes.

In at least one embodiment, the interconnection substrate may be formed by a lift-off method in which a resist pattern is formed and then metal is deposited in a vacuum and then the resist pattern is dissolved by using a solution.

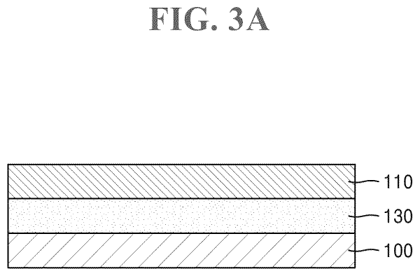

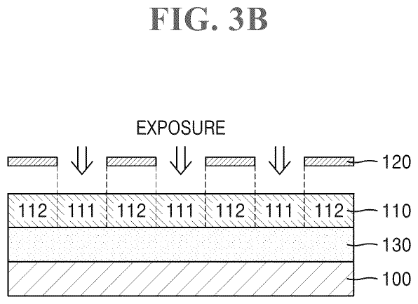

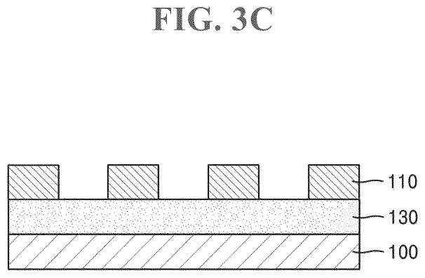

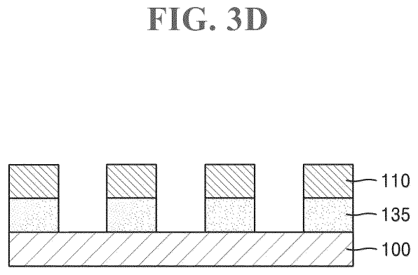

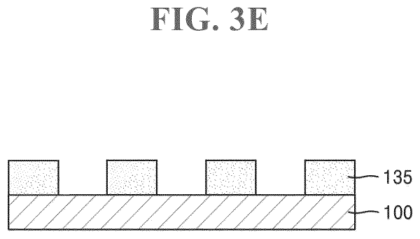

FIGS. 3A to 3E are each a cross-sectional view illustrating a method of forming a patterned structure, according to at least one embodiment.

Referring to FIG. 3A, a material layer 130 may be formed on the substrate 100 before forming the resist film 110 on the substrate 100. The resist film 110 may be formed on top of the material layer 130. The material layer 130 may include an insulating material (e.g., silicon oxide, silicon nitride), a semiconductor material(e.g., silicon), or a metal (e.g., copper). In at least one embodiment, the material layer 130 may be a multi-layer structure. A material for forming the material layer 130 may be different from a material for forming the substrate 100. In at least one embodiment, the resist film 110 may undergo a pre-exposure bake process.

Referring to FIG. 3B, exposure process to be exposed with high energy rays through a mask 120, after which the resist film 110 may include exposed regions 111 and unexposed regions 112.

Referring to FIG. 3C, the exposed resist film 110 may be developed by using a developer (e.g., a developing solution). The exposed region 111 may be washed away by the developer, whereas the unexposed region 112 may remain without being washed away by the developer.

Referring to FIG. 3D, exposed portions of the material layer 130 may be etched using the resist pattern 110 as a mask to form a material pattern 130 on the substrate 100.

Referring to FIG. 3E, the resist pattern 110 may be removed.

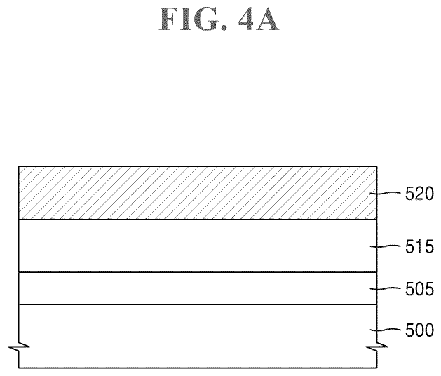

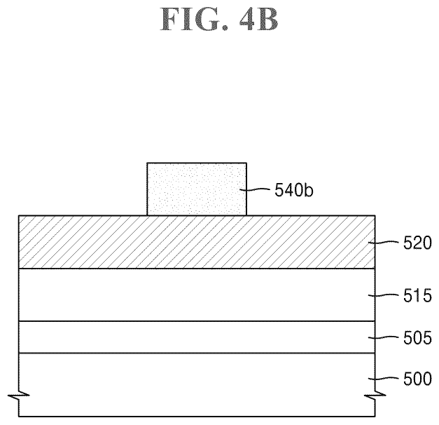

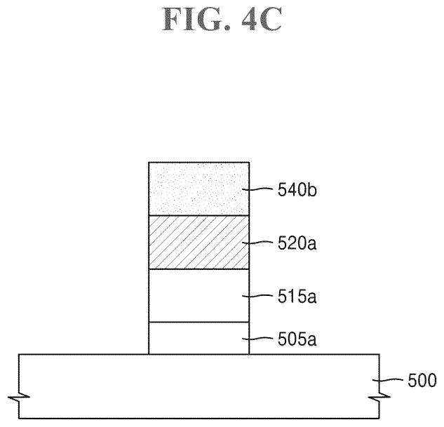

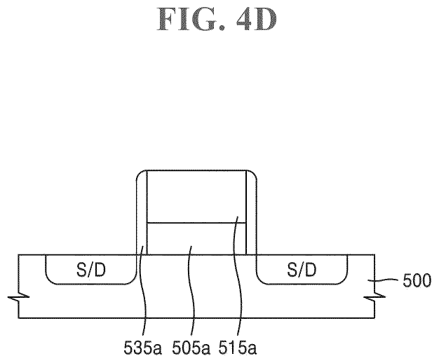

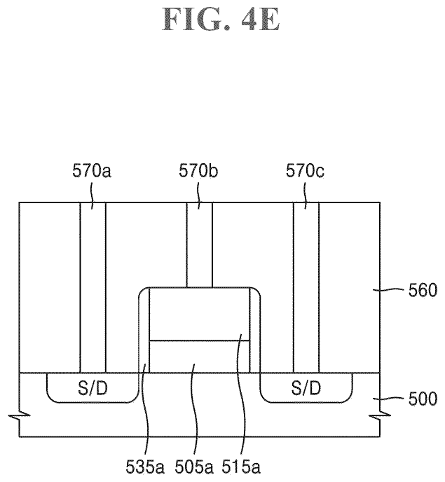

FIGS. 4A to 4E are each a cross-sectional view illustrating a method of manufacturing a semiconductor device, according to at least one embodiment.

Referring to FIG. 4A, a gate dielectric 505 (e.g., silicon oxide) may be formed on a substrate 500. The substrate 500 may be a semiconductor substrate, such as a silicon substrate. A gate layer 515 (e.g., doped polysilicon) may be formed on the gate dielectric 505. A hardmask layer 520 may be formed on the gate layer 515.

Referring to FIG. 4B, a resist pattern 540b may be formed on the hardmask layer 520. The resist pattern 540b may be formed by using the resist composition according to at least one of the embodiments described above.

Referring to FIG. 4C, the gate layer 515 and the gate dielectric 505 may be etched to form a hardmask pattern 520a, a gate electrode pattern 515a, and a gate dielectric pattern 505a.

Referring to FIG. 4D, a spacer layer (e.g., silicon nitride) may be formed over the gate electrode pattern 515a and the gate dielectric pattern 505a. The spacer layer may be formed by using a deposition process (e.g., CVD). The spacer layer may be etched to form spacers 535a on sidewalls of the gate electrode pattern 515a and the gate dielectric pattern 505a. After forming the spacers 535a, ions may be provided into the substrate 500 to form source/drain (S/D) impurity regions.

Referring to FIG. 4E, on the substrate 500, an interlayer insulating layer 560 (e.g., oxide) covering the gate electrode pattern 515a, the gate dielectric pattern 505a, and the spacers 535a may be formed. Then, electrical contacts 570a, 570b, and 570c may be formed in the interlayer insulating layer 560 to connect to the gate electrode 515a and the S/D regions. The electrical contacts 570a, 570b, and 570c may be formed of a conductive material (e.g., a metal). Although not illustrated, a barrier layer may be formed between the sidewalls of the interlayer insulating layer 560 and the electrical contacts 570a, 570b, and 570c.

FIGS. 4A to 4E illustrate an example of forming a transistor, but embodiments are not particularly limited thereto.

The resist composition according to at least one embodiment may be used in a patterning process to form other types of semiconductor devices.

The disclosure will be described in more detail with reference to Examples and Comparative Examples below, but the technical scope of the disclosure is not limited thereto.

### EXAMPLES

### Synthesis Example 1: Synthesis of Compound 1

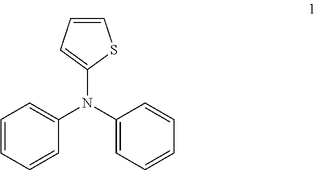 [SMILES: c1ccc(N(c2ccccc2)c2cccs2)cc1]

1.16 g (7.1 mmol) of 2-bromothiophene, 1.0 g (5.9 mmol) of diphenylamine, 0.17 g (0.3 mmol) of Pd(dba)₂, 0.24 g (0.6 mmol) of tert-Bu₃P, and 1.70 g (17.7 mmol) of t-BuONa were dissolved in 15 mL of toluene for a reaction at 110° C. for 24 hours. After completion of the reaction, the resulting mixture was cooled and filtered to remove impurities therefrom. Then, the filtrate was distilled under reduced pressure to obtain a crude mixture. The crude mixture was purified by silica column to synthesize 640 mg (yield of 43%) of Compound 1. Compound 1 thus obtained was subjected to structural analysis by using ¹H-nuclear magnetic resonance (NMR).

¹H-NMR (500 MHz, CD₂Cl₂) δ=7.27 (t, 4H), 7.12 (d, 4H) 7.06 (dd, 3H), 6.93 (dd, 1H), 6.74 (d, 1H).

### Synthesis Example 2: Synthesis of Compound 2

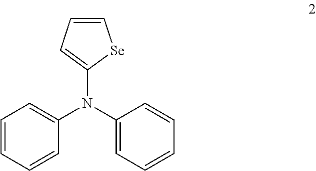 [SMILES: c1ccc(N(c2ccccc2)c2ccc[se]2)cc1]

700 mg (yield of 40%) of Compound 2 was synthesized in the same manner as in Synthesis Example 1, except that 1.82 g (7.1 mmol) of 2-iodoselenophene was used instead of 2-bromothiophene. Compound 2 thus obtained was subjected to structural analysis by using ¹H-NMR.

¹H-NMR (500 MHz, CD₂Cl₂) δ=7.68 (d, 1H), 7.28 (dd, 4H), 7.19 (d, 4H), 7.08 (m, 3H), 6.79 (d, 1H).

### Synthesis Example 3: Synthesis of Compound 3

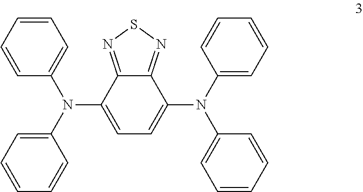 [SMILES: c1ccc(N(c2ccccc2)c2ccc(N(c3ccccc3)c3ccccc3)c3nsnc23)cc1]

2.94 g (10.0 mmol) of 4,7-dibromobenzo[c][1,2,5]thiadiazole, 3.72 g (22.0 mol) of diphenylamine, 0.58 g (1.0 mmol) of Pd(dba)₂, 0.81 g (2.0 mmol) of tert-Bu₃P, and 5.77 g (60.0 mmol) of t-BuONa were dissolved in 50 mL of toluene for a reaction at 110° C. for 48 hours. After completion of the reaction, the resulting mixture was cooled and filtered to remove impurities therefrom. Then, the filtrate was distilled under reduced pressure to obtain a crude mixture. The crude mixture was purified by silica column to synthesize 3.0 g (yield of 65%) of Compound 3. Compound 3 thus obtained was subjected to structural analysis by using ¹H-NMR.

¹H-NMR (500 MHz, CD₂Cl₂) δ=7.29 (t, 8H), 7.14 (s, 2H), 7.07 (dd, 12H)

### Synthesis Example 4: Synthesis of Compound 4

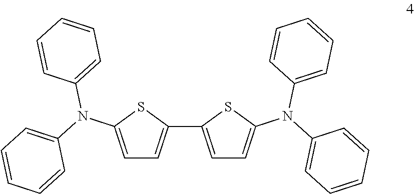 [SMILES: c1ccc(N(c2ccccc2)c2ccc(-c3ccc(N(c4ccccc4)c4ccccc4)s3)s2)cc1]

850 mg (yield of 34%) of Compound 4 was synthesized in the same manner as in the same manner as in Synthesis Example 3, except that 1.62 g (5 mmol) of 5,5′-dibromo-2,2′-bithiophene was used instead of 4,7-dibromobenzo[c][1,2,5]thiadiazole. Compound 4 thus obtained was subjected to structural analysis by using ¹H-NMR.

¹H-NMR (500 MHz, CD₂Cl₂) δ=7.31 (t, 8H), 7.15 (d, 8H), 7.06 (t, 4H), 6.86 (s, 2H), 6.57 (s, 2H).

### Synthesis Example 4: Synthesis of Polymer P-1

4-acetoxystyrene (AHS) (1.12 g, 0.007 mol) and 1-ethylcyclopentyl methacrylate (ECP) (1.26 g, 0.007 mol) were added to a 100 ml reactor. Here, 0.159 g of V601 was added as an initiator, and 7 mL of 1,4-dioxane was added as a solvent. The reactants were subjected to nitrogen bubbling, and then the reactor was sealed to allow a reaction at 80° C. for 4 hours. After completion of the reaction, the reaction product was precipitated by using methanol, and filtered to obtain a precipitate. The precipitate was dried in a vacuum oven at 40° C. After dispersing 2 g of the dried polymer in 16 g of methanol, 1.8 g of sodium methoxide solution (25 wt %, methanol) was added thereto. Following a reaction at room temperature for at least 4 hours, the resulting product was quenched in water. After neutralizing with acetic acid, the resulting product was precipitated by using methanol to obtain a precipitate. The precipitate was dried to obtain 1.2 g (yield of 40%) of a final product, Polymer P-1. Here, Polymer P-1 had a weight average molecular weight of 5,435 g/mol and PDI of 1.4. Also, as a result of structural analysis of Polymer P-1 thus obtained by using ¹H-NMR, the molar ratio of AHS:ECP was confirmed to be 47:53.

### Evaluation Example 1: Evaluation of Thin Film Phenomenon

100 parts by weight of Polymer P-1 of Synthesis Example 7, 35 parts by weight of PAG, 22.5 parts by weight of PDQ, and an amine compound shown in Table 2 were added to a casting solvent (PGME/PGMEA=7/3 (w/w)), and the mixed solution was filtered through a 0.2 μm separator filter. A hexamethyldisilazane (HMDS)-treated silicon wafer was spin-coated with the filtered casting solution at a speed of 1,500 rpm, and then dried (by PAB) at 110° C. for 1 minute to form a film. Next, EUV (ASML NXE-3350) was irradiated on a mask with a contact hole (hereinafter referred to as ‘CH’) pattern while changing the exposure dose and focus (exposure dose pitch: 1 mJ/cm², focus pitch: 0.020 m). Next, drying (by PEB) was performed thereon at 90° C. for 60 seconds, followed by immersing in a 2.38 wt % TMAH aqueous solution for 60 seconds, washing with deionized (DI) water for 10 seconds, and drying, thereby forming a resist pattern. For the resist pattern, the sensitivity (optimal exposure dose, Eₒₚ), critical dimension (CD), and critical dimension in-point uniformity (CD IPU) were each measured by using a critical dimension measurement scanning electron microscope (CD-SEM), and the results are shown in Table 2.

### (1) Sensitivity

(The optimal exposure dose Eₒₚ(mJ/cm²) at which CH patterns are obtained was measured and used as the sensitivity. The smaller this value is, the higher the sensitivity is.

### (2) CD IPU

By measuring the width ofmultiple CH patterns and the spacing between the CH patterns, the CD was calculated. The dispersion of the CH patterns was calculated therefrom to determine the CD IPU.

TABLE 2

| | | Content of | | | | |
|---|---|---|---|---|---|---|
| | | amine | | | | |
| | | compound | | CD | CD | CD |
| No. of | Amine | (parts by | Eₒₚ | Min | Max | IPU |
| Example | compound | weight) | (mJ/cm²) | (min) | (nm) | (nm) |
| Example 1 | Compound 1 | 15 | 60.2 | 18.5 | 20.3 | 3.32 |
| Comparative | — | 0 | 64.0 | 17.5 | 20.3 | 3.42 |
| Example 1 | | | | | | |
| Comparative | Triphenylamine | 15 | 61.3 | 18.1 | 20.7 | 3.34 |
| Example 2 | | | | | | |
| Comparative | Triphenylamine | 20 | 60.6 | 18.0 | 20.7 | 3.35 |
| Example 3 | | | | | | |
| Comparative | Triphenylamine | 30 | 61.7 | 19.1 | 20.3 | 3.32 |
| Example 4 | | | | | | |
| Comparative | Triphenylamine | 40 | 60.6 | 19.4 | 20.1 | 3.30 |
| Example 5 | | | | | | |
| Comparative | ST005 | 15 | 66.8 | 19.5 | 19.5 | 3.32 |
| Example 6 | | | | | | |

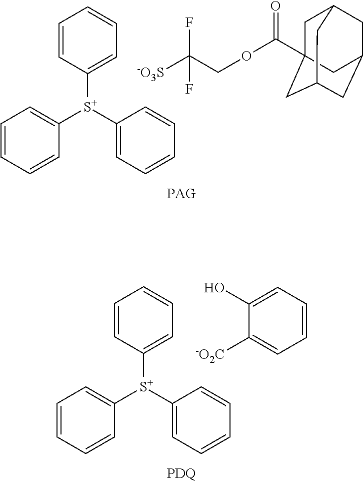

*ST005 is a commercially available sensitizer.

Referring to Table 2, it was confirmed that the Example 1 had improved sensitivity compared to Comparative Examples 1 to 6. It was also confirmed that Example 1 had improved CD IPU compared to Comparative Examples 1 to 5. However, it was confirmed that Example 1 had a similar level of CD IPU as Comparative Example 6. In other words, it was confirmed that the resist composition including the amine compound of Example 1 had improved sensitivity and/or was able to form a more uniform pattern.

According to the one or more embodiments, a resist composition having improved sensitivity, improved resolution, and/or reduced defects may be provided.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An amine compound represented by Formula 1: 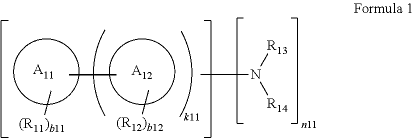
wherein, in Formula 1,
N is nitrogen,
A₁₁ is one of i) a first ring, ii) a condensed ring in which at least two first rings are condensed with each other, or iii) a condensed ring in which at least one first ring and at least one second ring are condensed with each other,
A₁₂ is one of i) a single bond, ii) a first ring, iii) a second ring, iv) a condensed ring in which at least two first rings are condensed with each other, v) a condensed ring in which at least two second rings are condensed with each other, or vi) a condensed ring in which at least one first ring and at least one second ring are condensed with each other,
k11 is an integer from 0 to 3,
n11 is an integer from 1 to 4,
R₁₁ and R₁₂ are each independently at least one of hydrogen, deuterium, a halogen, a cyano group, a nitro group, a hydroxyl group, a carboxylate group, an ester moiety, a substituted or unsubstituted C₁-C₃₀ alkyl group, a substituted or unsubstituted C₃-C₃₀ cycloalkyl group, a substituted or unsubstituted C₁-C₃₀ alkoxy group, or a substituted or unsubstituted C₃-C₃₀ cycloalkoxy group,
R₁₃ and R₁₄ are each independently a substituted or unsubstituted C₆-C₃₀ aryl group or a substituted or unsubstituted C₁-C₃₀ heteroaryl group, and
b11 and b12 are each independently an integer from 1 to 8, and
wherein the first ring is a 5-membered ring including at least one of S, Se, and Te, and the second ring is a 4- to 10-membered ring optionally including a heteroatom.

2. The amine compound of claim 1, wherein the first ring is represented by Formula 4: 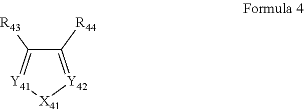
wherein, in Formula 4,
X₄₁ is one of S, Se, or Te,
Y₄₁ is one of N or CR₄₁,
Y₄₂ is one of N or CR₄₂,
R₄₁ to R₄₄ are each independently at least one of *—N(R₁₃)(R₁₄), hydrogen, deuterium, a halogen, a cyano group, a nitro group, a hydroxyl group, a carboxylate group, an ester moiety, a substituted or unsubstituted C₃-C₃₀ alkyl group, a substituted or unsubstituted C₃-C₃₀ cycloalkyl group, a substituted or unsubstituted C₃-C₃₀ alkoxy group, or a substituted or unsubstituted C₃-C₃₀ cycloalkoxy group, and
R₁₃ and R₁₄ are each independently a substituted or unsubstituted C₆-C₃₀ aryl group or a substituted or unsubstituted C₁-C₃₀ heteroaryl group.

3. The amine compound of claim 1, wherein the second ring is one of tetrahydropyrane, dihydropyrane, pyrane, tetrahydrothiopyrane, dihydrothiopyrane, thiopyrane, tetrahydrofurane, dihydrofurane, piperidine, tetrahydropyridine, dihydropyridine, pyrrolidine, dihydropyrrole, pyrrole, imidazole, pyrazole, furan, oxazole, pyridine, pyrazine, pyridazine, pyrimidine, triazine, cyclopentane, cyclopentadiene, cyclohexane, cyclohexene, cyclohexadiene, benzene, or naphthalene.

4. The amine compound of claim 1, wherein A₁₁ and A₁₂ are each independently i) the first ring, ii) a condensed ring in which at least two of the first rings are condensed with each other, or iii) a condensed ring in which at least one of the first ring and at least one of the second ring are condensed with each other.

5. The amine compound of claim 1, wherein
A₁₁ and A₁₂ are each independently represented by at least one of Formulae 4-11 to 4-26:

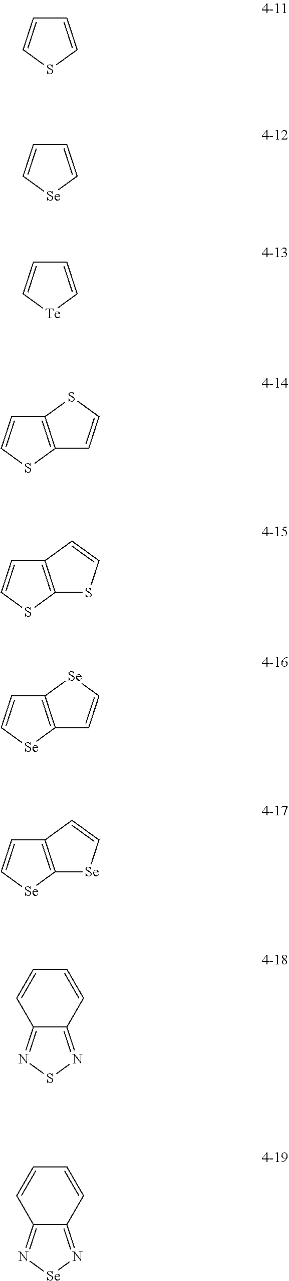

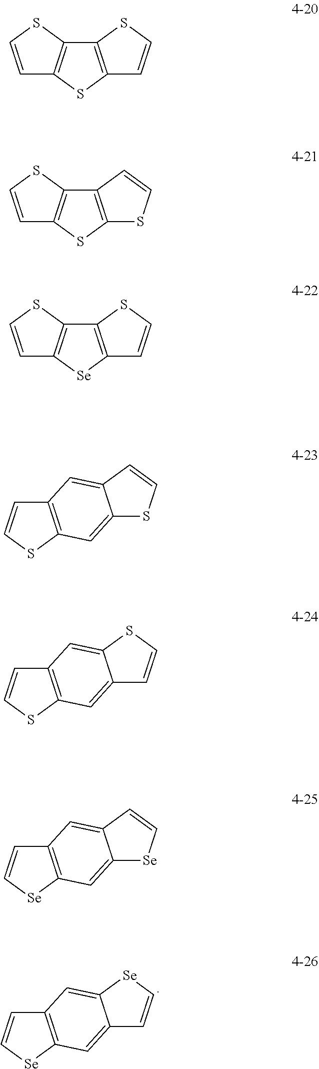

6. The amine compound of claim 1, wherein
R₁₁ and R₁₂ are each independently selected from: hydrogen; deuterium; a halogen; a hydroxyl group; a carboxylate group; an ester moiety; and a C₁-C₃₀ alkyl group, a C₃-C₃₀ cycloalkyl group, a C₁-C₃₀ alkoxy group, and a C₃-C₃₀ cycloalkoxy group, each unsubstituted or substituted with deuterium, a halogen, a hydroxyl group, a carboxylate group, an ester moiety, or any combination thereof.

7. The amine compound of claim 1, wherein R₁₃ and R₁₄ are each independently a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, a halogen, a hydroxyl group, a carboxylate group, an ester moiety, a C₁-C₂₀ alkyl group, a C₁-C₂₀ alkoxy group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindole group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or a combination thereof.

8. The amine compound of claim 1, wherein the amine compound is represented by one of Formulae 1-1 to 1-4:

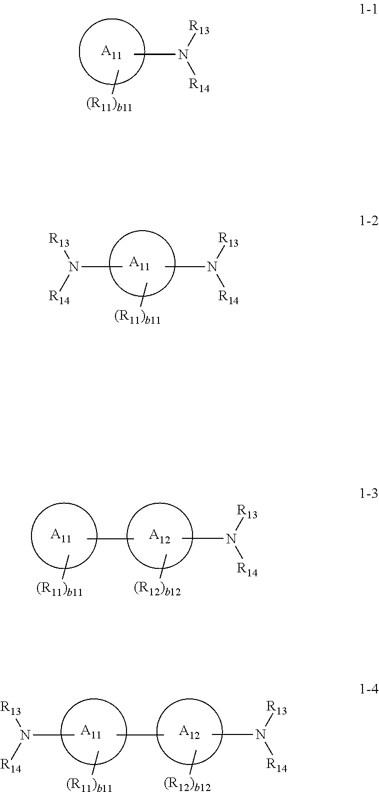

9. The amine compound of claim 1, wherein the amine compound is at least one of Group I:

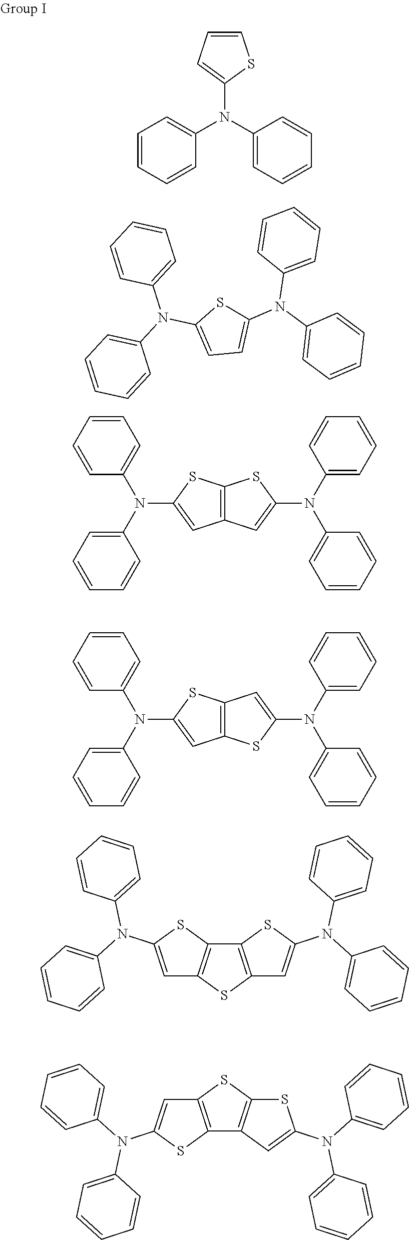

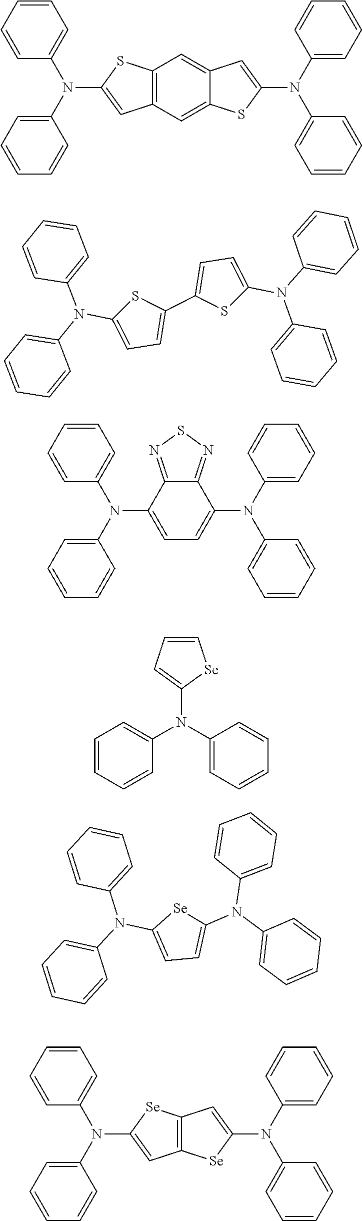

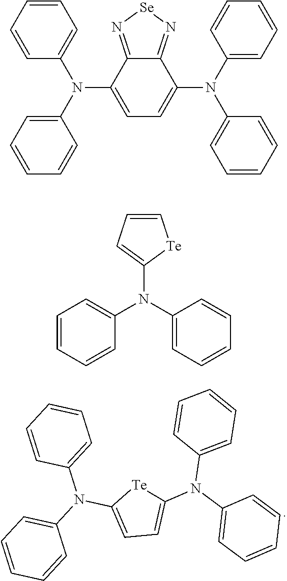

10. A resist composition comprising:
the amine compound of claim 1;
a polymer;
a photoacid generator; and
an organic solvent.

11. The resist composition of claim 10, wherein an amount of the amine compound is in a range of about 0.1 parts by weight to about 50 parts by weight, based on 100 parts by weight of the resist composition.

12. The resist composition of claim 10, wherein the polymer comprises at least one of a first repeating unit represented by Formula 2 and a second repeating unit represented by Formula 3: 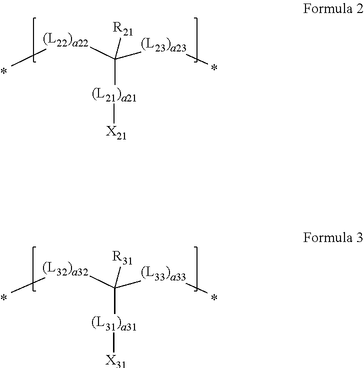
wherein, in Formulae 2 and 3,
L₂₁ to L₂₃ and L₃₁ to L₃₃ are each independently a single bond; O; S; C(═O); C(═O)O;
OC(═O); C(═O)NH; NHC(═O); S(═O); S(═O)₂O; OS(═O)₂; or a linear, branched, or cyclic C₁-C₃₀ divalent hydrocarbon group that optionally includes a heteroatom,
a21 to a23 and a31 to a33 are each independently an integer from 1 to 4,
R₂₁ and R₃₁ are each independently at least one of hydrogen; deuterium; a halogen; a cyano group; a hydroxyl group; an amino group; a carboxylate group; a thiol group; a carbonyl moiety; an ester moiety; a sulfonate moiety; a carbonate moiety; a lactone moiety; a sultone moiety; a carboxylic anhydride moiety; or a linear, branched, or cyclic C₁-C₃₀ monovalent hydrocarbon group that optionally includes a heteroatom,
X₂₁ is an acid labile group,
X₃₁ is a non-acid labile group, and
* indicates a binding site to a neighboring atom.

13. The resist composition of claim 12, wherein
X₂₁ is represented by one of Formulae 6-1 to 6-12: 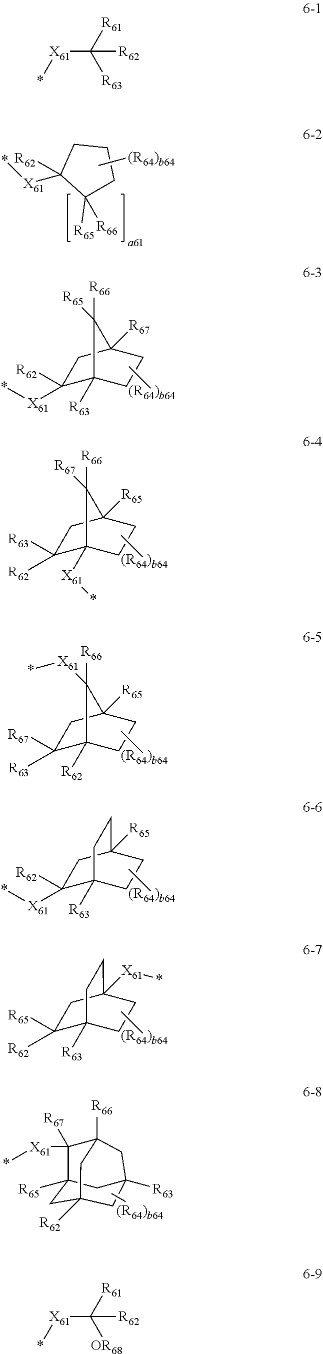 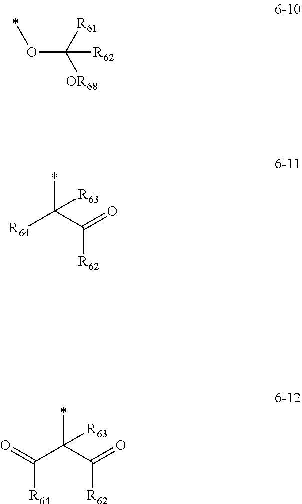
wherein, in Formulae 6-1 to 6-12,
X₆₁ is at least one of an ester moiety, a carbonate moiety, a carbamate moiety, or a sulfonate moiety,
a61 is an integer from 0 to 6,
R₆₁ and R₆₈ are each independently a linear, branched or cyclic C₁-C₂₀ monovalent hydrocarbon group that optionally includes a heteroatom,
R₆₂ to R₆₇ are each independently: hydrogen; deuterium; a halogen; a cyano group; a hydroxyl group; an amino group; a carboxylate group; a thiol group; a carbonyl moiety; an ester moiety; a sulfonate moiety; a carbonate moiety; a carbamate moiety; a lactone moiety; a sultone moiety; a carboxylic anhydride moiety; or a linear, branched, or cyclic C₁-C₃₀ monovalent hydrocarbon group that optionally includes a heteroatom,
two adjacent groups among R₆₁ to R₆₈ are optionally bonded to each other to form a ring,
b64 is an integer from 1 to 10, and
* indicates a binding site to a neighboring atom.

14. The resist composition of claim 12, wherein X₃₁ is hydrogen; a halogen; a cyano group; a hydroxyl group; a carboxylate group; a thiol group; an amino group; or a linear, branched, or cyclic C₁-C₃₀ monovalent hydrocarbon group that optionally includes at least one polar moiety selected from a halogen, a cyano group, a hydroxyl group, a thiol group, a carboxylate group, O, C═O, C(═O)O, OC(═O), S(═O)₂O, OS(═O)₂, a lactone moiety, a sultone moiety, and a carboxylic anhydride moiety.

15. The resist composition of claim 10, wherein the photoacid generator is represented by Formula 7:
B₇₁⁺A₇₁⁻ Formula 7
wherein, in Formula 7,
B₇₁⁺ is represented by Formula 7A, and A₇₁⁻ is represented by one of Formulae 7B to 7D, and
B₇₁⁺ and A₇₁⁻ are optionally linked to each other via a carbon-carbon covalent bond: 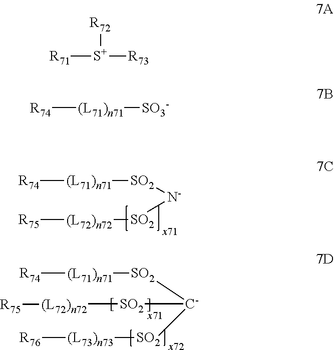
wherein, in Formulae 7A to 7D,
L₇₁ to L₇₃ are each independently a single bond or CRR′, and R and R′ are each at least one of independently hydrogen, deuterium, a halogen, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group, or a C₃-C₃₀ cycloalkoxy group,
n71 to n73 are each independently 1, 2, or 3,
x71 and x72 are each independently 0 or 1,
R₇₁ to R₇₃ are each independently a linear, branched, or cyclic C₁-C₃₀ monovalent hydrocarbon group that optionally includes a heteroatom,
two adjacent groups among R₇₁ to R₇₃ are optionally bonded to each other to form a condensed ring, and
R₇₄ to R₇₆ are each independently one of hydrogen; a halogen; or a linear, branched, or cyclic C₁-C₃₀ monovalent hydrocarbon group that optionally includes a heteroatom.

16. The resist composition of claim 10, further comprising:
a quencher.

17. The resist composition of claim 16, wherein the quencher is represented by Formula 8:
B₈₁⁺A⁸¹⁻ Formula 8
wherein, in Formula 8,
B₈₁⁺ is represented by one of Formulae 8A to 8C, and A₈₁⁻ is represented by one of Formulae 8D to 8F, and
B₈₁⁺ and A₈₁⁻ are optionally linked to each other via a carbon-carbon covalent bond: 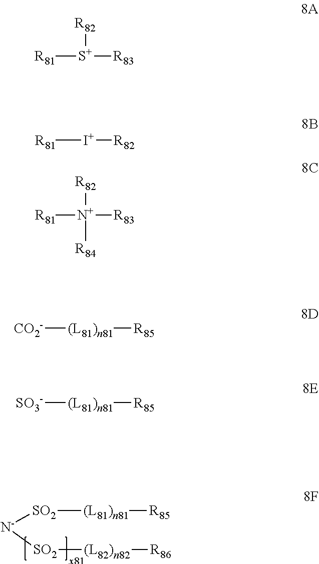
wherein, in Formulae 8A to 8F,
L₈₁ and L₈₂ are each independently a single bond or CR₁R₁′, and R₁ and R₁′ are each at least one of independently hydrogen, deuterium, a halogen, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group, or a C₃-C₃₀ cycloalkoxy group,
n81 and n82 are each independently 1, 2, or 3,
x81 is 0 or 1,
R₈₁ to R₈₄ are each independently a linear, branched, or cyclic C₁-C₃₀ monovalent hydrocarbon group that optionally includes a heteroatom,
two adjacent groups among R₈₁ to R₈₄ are optionally bonded to each other to form a condensed ring, and
R₈₅ and R₈₆ are each independently one of hydrogen; a halogen; or a linear, branched, or cyclic C₁-C₃₀ monovalent hydrocarbon group that optionally includes a heteroatom.

18. A method of forming a pattern, the method comprising:
forming a resist film by applying the resist composition of claim 10 onto a substrate;
exposing at least a portion of the resist film to high-energy rays to generate an exposed resist film; and
developing the exposed resist film using a developer.

19. The method of claim 18, wherein the high-energy rays include at least one of ultraviolet rays, deep ultraviolet (DUV) rays, extreme ultraviolet (EUV) rays, X rays, γ rays, electron beam (EB) rays, or α rays.

20. The method of claim 18, wherein
the exposed resist film includes an exposed region and an unexposed region, and
the unexposed region is removed by the developing.
